(19) United States
(12) Patent Application Publication
(10) Pub. No.: US 2009/0181008 A1
(43) Pub. Date: Jul. 16, 2009

(54) METHODS OF TREATING ALZHEIMER'S DISEASE

(75) Inventors: Sandip Ray, San Francisco, CA (US); Anton Wyss-Coray, Belmont, CA (US)

Correspondence Address: MORRISON & FOERSTER LLP, 755 PAGE MILL RD, PALO ALTO, CA, 94304-1018, US

(73) Assignee: Satoris, Inc.

(21) Appl. No.: 12/093,431

(22) PCT Filed: Nov. 13, 2006
(86) PCT No.: PCT/US06/44198

§ 371 Date: Nov. 24, 2008

Related U.S. Application Data
(60) Provisional application No. 60/735,552, filed on Nov. 10, 2005.

(51) Int. Cl. A61K 39/395; A61K 38/02; A61P 25/28

(52) U.S. Cl. 424/130.1; 514/2

(57) ABSTRACT

The invention provides biomarkers that are modulated in Alzheimer's disease including IL-1α, PDGF-BB, TNF-α, M-CSF, G-CSF, GNDF, eotaxin 2, MCP-3, PARC, AgRP, MSP-α, and BTC. Described are methods for preventing, treating, alleviating symptoms of, or delaying the development of Alzheimer's Disease (AD) in an individual diagnosed with Alzheimer's Disease or at risk for developing the disease by modulating the biological activity of, or the levels of any one or more of these AD-associated biomarkers. Modulation of biomarker levels by administration of biomarker proteins, biologically active fragments thereof, agonists, antagonists and antibodies are provided.

### CROSS-REFERENCE TO RELATED APPLICATIONS

This application claims benefit of U.S. Provisional Patent Application No. 60/735,552, filed Nov. 10, 2005, which is incorporated by reference herein in its entirety.

### FIELD OF INVENTION

The present invention relates generally to methods and compositions for the treatment and prevention of Alzheimer's disease. More specifically, the present invention relates to methods for the treatment of Alzheimer's disease by administration of polypeptide factors (products), that have been identified as Alzheimer's disease-associated biomarkers; agonists and/or antagonists of the biomarkers; and agonist and/or antagonists of the receptors of the biomarkers resulting in amelioration of or delay in progress of symptoms related to Alzheimer's disease.

### BACKGROUND OF THE INVENTION

An estimated 4.5 million Americans have Alzheimer's Disease (“AD”). By 2050, the estimated prevalence of Alzheimer's disease will range from 11.3 million to 16 million individuals. Currently, the societal cost of Alzheimer's disease to the U.S. alone is $100 billion per year, including $61 billion borne by U.S. businesses. Neither Medicare nor most private health insurance companies cover the long-term care that most patients require. Alzheimer's disease occurs throughout the world and accounts for one-half to two-thirds of all cases of late-life intellectual failure in developed countries having populations with high life expectancies.

Alzheimer's disease is a devastating, progressive dementia characterized by memory failure, amnesia, disturbances in emotional behavior and difficulty in managing spatial relationships or motor skills. The disease is diagnosed mainly by clinical symptoms, after other causes of dementia have been excluded. After death, a diagnosis can be conclusively established by the observation by several main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites). These characteristic changes are found in the hippocampus, cerebral cortex, and other areas of the brain essential for cognitive function. Plaques are formed mostly from the extracellular deposition of amyloid beta (“Aβ”), a peptide derived from proteolytic cleavage of amyloid precursor protein (“APP”). Neurofibrillary tangles are abnormal intracellular cytoskeletal filaments formed from paired helical filaments composed of neurofilament and hyperphosphorylated tau protein, which is a microtubule-associated protein. It is not clear, however, whether these pathological changes are only associated with the disease or truly involved in the degenerative process. Late-onset/sporadic Alzheimer's disease has virtually identical pathology to inherited early-onset/familial Alzheimer's disease (FAD), suggesting common pathogenic pathways for both forms of Alzheimer's disease. To date, genetic studies have identified three genes that when mutated cause autosomal dominant, early-onset Alzheimer's disease, amyloid precursor protein (“APP”), presenilin 1 (“PS1”), and presenilin 2 (“PS2”). A fourth gene, apolipoprotein E (“ApoE”), is the strongest and most common genetic risk factor for Alzheimer's disease, but does not necessarily cause it. All mutations associated with APP and PS proteins can lead to an increase in the production of Aβ peptides, specifically the more amyloidogenic form, Aβ₄₂. In addition to genetic influences on amyloid plaque and intracellular tangle formation, other cellular factors (e.g., cytokines, neurotoxins, etc.) and environmental factors may also play important roles in the development and progression of Alzheimer's disease.

The main clinical feature of Alzheimer's disease is a progressive cognitive decline leading to memory loss. The memory dysfunction involves impairment of learning new information which is often characterized as short-term memory loss. In the early (mild) and moderate stages of the illness, recall of remote well-learned material may appear to be preserved, but new information cannot be adequately incorporated into memory. In addition, disorientation in regard to time is closely related to memory disturbance. In a typical case of Alzheimer's disease, the onset is so insidious that family members have difficulty estimating when the impairment began.

Language impairments are also a prominent part of Alzheimer's disease. Early on these often manifest as “word finding” difficulties in normal conversational speech. As the disease progresses, the language of the Alzheimer's disease patient is often vague, lacking in specifics and may have increased automatic phrases and clichés. Difficulty in naming everyday objects is often prominent. Complex deficits in visual function are present in many Alzheimer's disease patients, as are other focal cognitive deficits such as apraxia, acalculia and left-right disorientation. Impairments of judgment and problem solving are also frequently seen.

Non-cognitive or behavioral symptoms are also common in Alzheimer's disease and may account for an even larger proportion of caregiver burden or stress than the cognitive dysfunctions. Personality changes are commonly reported and range from progressive passivity to marked agitation. Patients may exhibit changes such as decreased expressions of affection. Depressive symptoms are present in up to 40% of the patients with a similar rate for symptoms of anxiety. Psychosis occurs in 25% of the patients and in some cases, personality changes may predate cognitive abnormality.

Presently available pharmaceutical therapy for treatment of Alzheimer's disease is directed almost entirely to disease symptoms, providing only temporary or partial clinical benefit. Therapy for Alzheimer's disease is divided into three main categories: 1) control of abnormal behavior associated with the illness, 2) attempts to restore cognitive function, and 3) attempts to delay cognitive decline. Behavioral disturbances such as agitation, insomnia, wandering, suspiciousness, hallucinations, and hostility often arise during the course of dementia. Psychotic symptoms are generally treated with low-dose neuroleptic medication. Although some pharmaceutical agents have been described that offer partial symptomatic relief, no comprehensive pharmacological therapy is currently available for the treatment of Alzheimer's disease. Therefore, new and effective therapeutics and therapies are needed for treatment of Alzheimer's disease.

Other references of interest include U.S. Patent Application Publication No. 20050221348; PCT publication WO 05/052592; Fiala et al. (1998) Mol. Med. 4:480-9; and Fiala et al (2005) J. Alzheimer's Dis. 7:221-32; all of which are specifically incorporated herein by reference.

All references cited herein, including patent applications and publications are incorporated by reference in their entirety.

### BRIEF SUMMARY OF THE INVENTION

Provided herein are methods of treating or preventing Alzheimer's disease (AD) in an individual comprising modulating the biological activity of, and/or the levels of, any one or more AD-associated biomarker(s) selected from the group consisting of, or alternatively, any one or more of, interleukin-1α (IL-1α), platelet-derived growth factor-BB (PDGF-BB), tumor necrosis factor-α (TNF-α), macrophage colony-stimulating factor (M-CSF), granulocyte colony-stimulating factor (G-CSF), glial cell line-derived neurotrophic factor (GNDF), eotaxin 2, monocyte chemotactic protein-3 (MCP-3), pulmonary and activation-regulated chemokine (PARC), Agouti-related protein (AgRP), macrophage stimulating protein-α (MSP-α), and betacellulin (BTC). In some embodiments, treating comprises alleviating at least one symptom of Alzheimer's disease.

In some embodiments, the methods comprise increasing a level of at least one AD-associated biomarker selected from IL-1α, PDGF-BB, TNF-α, M-CSF, G-CSF, GDNF, eotaxin 2, and/or MCP-3 in a biological sample from an individual. In some embodiments, the methods comprise increasing a level of at least two AD-associated biomarkers selected from IL-1α, PDGF-BB, TNF-α, M-CSF, G-CSF, GDNF, eotaxin 2, and/or MCP-3. In some embodiments, the methods comprise increasing at least three AD-associated biomarkers selected from IL-1α, PDGF-BB, TNF-α, M-CSF, G-CSF, GDNF, eotaxin 2, and/or MCP-3. In some embodiments, the methods comprise increasing a biological activity of at least one AD-associated biomarker selected from IL-1α, PDGF-BB, TNF-α, M-CSF, G-CSF, GDNF, eotaxin 2, and/or MCP-3 or increasing a biological activity of at least one receptor of IL-1α, PDGF-BB, TNF-α, M-CSF, G-CSF, GDNF, eotaxin 2, and/or MCP-3. In some examples the biological sample is a peripheral biological fluid sample. In some embodiments the peripheral biological fluid sample is blood, plasma or serum.

In some embodiments, the methods comprise decreasing a level of at least one AD-associated biomarker selected from PARC, AgRP, MSP-α and/or BTC in a biological sample from an individual. In some embodiments, the methods comprise decreasing a level of at least two AD-associated biomarker selected from PARC, AgRP, MSP-α and/or BTC. In some embodiments, the methods comprise decreasing a level of at least three AD-associated biomarkers selected from PARC, AgRP, MSP-α and/or BTC. In some embodiments, the methods comprise decreasing a biological activity of at least one AD-associated biomarker selected from PARC, AgRP, MSP-α and/or BTC. In some embodiments, the methods comprise decreasing a biological activity of at least one receptor of PARC, AgRP, MSP-α and/or BTC. In some embodiments the biological sample is a peripheral biological fluid sample. In some embodiments the peripheral biological fluid sample is blood, plasma or serum.

Provided herein are methods for treating or preventing Alzheimer's disease in an individual, said method comprising administering a therapeutically effective amount of a composition to said individual comprising at least one substance selected from a) a polypeptide of IL-1α, PDGF-BB, TNF-α, M-CSF, G-CSF, GDNF, eotaxin 2, or MCP-3; b) a fragment or variant of IL-1α, PDGF-BB, TNF-α, M-CSF, G-CSF, GDNF, eotaxin 2, or MCP-3 that retains a biological activity; c) an agonist of IL-1α, PDGF-BB, TNF-α, M-CSF, G-CSF, GDNF, eotaxin 2, or MCP-3; d) an agonist of a receptor of IL-1α, PDGF-BB, TNF-α, M-CSF, G-CSF, GDNF, eotaxin 2, or MCP-3; e) an antagonist of PARC, AgRP, MSP-α or BTC; f) an antagonist of a receptor of PARC, AgRP, MSP-α or BTC; and a combination thereof. In some embodiments, the agonist is a small molecule, antibody, a biomarker mimic, a biomarker structural analog or a nucleic acid molecule. In some embodiments, the antagonist is a small molecule, an antibody, a biomarker structural analog or a nucleic acid molecule. In some embodiments, treating comprises alleviating at least one symptom of Alzheimer's disease.

In some embodiments, the composition comprises at least one polypeptide or fragment thereof, in an amount sufficient to result in a significant increase in a level of the polypeptide in a biological fluid sample from the individual. In some embodiments, the composition comprises at least one agonist, in an amount sufficient to result in a significant increase in a level of IL-1α, PDGF-BB, TNF-α, M-CSF, G-CSF, GDNF, eotaxin 2, or MCP-3 in a biological fluid sample from the individual. In some embodiments, the composition comprises at least one agonist, in an amount sufficient to result in a significant increase in a level of a receptor of IL-1α, PDGF-BB, TNF-α, M-CSF, G-CSF, GDNF, eotaxin 2, or MCP-3.

In some embodiments, the composition comprises at least one antagonist, in an amount sufficient to result in a significant decrease in a level of PARC, AgRP, MSP-α or BTC in a biological fluid sample from the individual. In some embodiments, the composition comprises at least one antagonist, in an amount sufficient to result in a significant decrease in a level of a receptor of PARC, AgRP, MSP-α or BTC.

Provided herein are methods for treating or preventing Alzheimer's disease in an individual, said method comprising administering to an individual a therapeutically effective amount of a composition comprising at least one antagonist of PARC, AgRP, MSP-α or BTC; at least one antagonist of a receptor of PARC, AgRP, MSP-α or BTC; or a combination thereof. In some embodiments, the antagonist is a small molecule, an antibody, a biomarker structural analog or a nucleic acid molecule. In some embodiments, treating comprises alleviating at least one symptom of Alzheimer's disease.

Provided herein are methods for treating or preventing Alzheimer's disease in an individual, the method comprising administering to the individual an agent which modulates monocyte/macrophage function in an amount sufficient to modulate at least one AD-associated biomarker selected from the group of IL-1α, TNF-α, M-CSF, eotaxin-2, MCP-3, PARC, and MSP-α. In some embodiments, treating comprises alleviating at least one symptom of Alzheimer's disease.

Provided herein are methods of delaying development of Alzheimer's disease in an individual, said method comprising administering to an individual a therapeutically effective amount of a composition comprising at least one substance selected from a) a polypeptide of IL-1α, PDGF-BB, TNF-α, M-CSF, G-CSF, GDNF, eotaxin 2, or MCP-3; b) a fragment or variant of IL-1α, PDGF-BB, TNF-α, M-CSF, G-CSF, GDNF, eotaxin 2, or MCP-3 that retains a biological activity; or c) an agonist of IL-1α, PDGF-BB, TNF-α, M-CSF, G-CSF, GDNF, eotaxin 2, or MCP-3; and a combination thereof. In some embodiments, the agonist is a small molecule, antibody, a biomarker mimic, a biomarker structural analog or a nucleic acid molecule.

Provided herein are methods of delaying development of Alzheimer's disease in an individual, said method comprising administering to an individual a therapeutically effective amount of at least one antagonist of PARC, AgRP, MSP-α or BTC; at least one antagonist of a receptor of PARC, AgRP, MSP-α or BTC; or a combination thereof. In some embodiments, the antagonist is a small molecule, an antibody, a biomarker structural analog or a nucleic acid molecule.

Provided herein are methods of treating Alzheimer's disease in an individual with at least one risk factor for Alzheimer's disease. Provided herein are methods of preventing or delaying development of Alzheimer's disease in an individual with at least one risk factor for Alzheimer's disease. In some embodiments, the risk factor is diagnosis of mild cognitive impairment, advanced age, family history, genetics, Down syndrome, history of head injury, exposure to environmental toxins and/or low education level. In some embodiments, treating comprises alleviating at least one symptom of Alzheimer's disease.

Provided herein are methods of diagnosing Alzheimer's disease in an individual, said method comprising, a) detecting, measuring, and/or identifying one or more of the biomarkers selected from the group consisting of IL-1α, PDGF-BB, TNF-α, M-CSF, G-CSF, GDNF, eotaxin 2, MCP-3, PARC, AgRP, MSP-α and BTC in a biological fluid sample from the individual; and/or b) comparing measured levels of any one or more of the biomarkers selected from the group consisting of IL-1α, PDGF-BB, TNF-α, M-CSF, G-CSF, GDNF, eotaxin 2, MCP-3, PARC, AgRP, MSP-α and BTC in a biological fluid sample from the individual to an appropriate control. In some embodiments the biological sample is a peripheral biological fluid sample. In some embodiments the peripheral biological fluid sample is blood, plasma or serum.

Provided herein are methods for delaying the development of Alzheimer's disease in an individual with Alzheimer's disease or at risk of developing Alzheimer's disease, the method comprising detecting in a biological sample an elevated level of at least one biomarker selected from PARC, AgRP, MSP-α and BTC, or a reduced level of at least one biomarker selected from IL-1α, PDGF-BB, TNF-α, M-CSF, G-CSF, GNDF, eotaxin-2 and MCP-3. In some embodiments the method comprises administering a therapeutically effective amount of a composition comprising at least one substance selected from: a polypeptide of IL-1α, PDGF-BB, TNF-α, M-CSF, G-CSF, GNDF, eotaxin-2 or MCP-3; a fragment or variant of IL-1α, PDGF-BB, TNF-α, M-CSF, G-CSF, GNDF, eotaxin-2 or MCP-3 that retains a biological activity; an agonist of IL-1α, PDGF-BB, TNF-α, M-CSF, G-CSF, GNDF, eotaxin-2 or MCP-3; an agonist of a receptor of IL-1α, PDGF-BB, TNF-α, M-CSF, G-CSF, GNDF, eotaxin-2 or MCP-3; an antagonist of PARC, AgRP, MSP-α or BTC; and an antagonist of a receptor of PARC, AgRP, MSP-α or BTC. In some embodiments, the detection comprises use of an antibody-based array. In some embodiments the antibody-based array comprises antibodies specific for one or more polypeptides selected from IL-1α, PDGF-BB, TNF-α, M-CSF, G-CSF, GNDF, eotaxin-2, MCP-3, PARC, AgRP, MSP-α and BTC. In some embodiments the biological sample is a peripheral biological fluid sample. In some embodiments the peripheral biological fluid sample is blood, plasma or serum.

### BRIEF DESCRIPTION OF THE DRAWINGS

FIG. 1 shows predictive analysis of microarray (PAM) data for 1-120 biomarkers. Shown are error rates for the training set, the cross-validation and the test set of samples from individuals diagnosed with Alzheimer's disease and samples from non-demented controls. TR refers to training set data; CV refers to cross-validation data; TE refers to test set data.

FIG. 2 shows functional clustering with twelve predictive biomarkers identified with PAM. Black indicates little or no relationship between a biomarker and a functional grouping. White indicates a decrease in levels of a biomarker as compared to a normal reference; cross-hatched indicates an increase in levels of a biomarker as compared to a normal reference.

### DETAILED DESCRIPTION OF THE INVENTION

Described herein are methods for treatment of Alzheimer's disease (AD) in an individual in need of treatment. Also described herein are methods for prevention of Alzheimer's disease in an individual with at least one risk factor for AD or a diagnosis of mild cognitive impairment. In general, the methods are based on the identification of a group of Alzheimer's disease-associated (AD-associated) biomarkers (shown herein in Tables 1-4) which can be used as predictors for Alzheimer's disease diagnosis. These biomarkers demonstrate either increased or decreased protein plasma levels in individuals diagnosed with AD as compared with controls (such as, for example, control individuals) as determined by antibody-based protein microarray analyses. It is believed that modulation of the levels of or biological activities of one or more of these biomarkers can be effective in treating Alzheimer's disease, and/or ameliorating the symptoms of AD, and/or improving the accompanying cognitive and/or memory deficits which are characteristic of AD, and/or delaying the development of AD, and/or delaying the progression of AD, and/or preventing AD, and/or decreasing the risk of onset of AD, and/or increasing the time to onset of AD.

The inventors have discovered a collection of biomarkers including interleukin 1α, platelet-derived growth factor beta chain, tumor necrosis factor-α, macrophage colony-stimulating factor, granulocyte colony-stimulating factor, glial cell line-derived neurotrophic factor, eotaxin 2, monocyte chemotactic protein 3, pulmonary and activation-regulated chemokine, agouti-related protein, macrophage stimulating protein-α and betacellulin, present in a biological fluid sample of individuals which are either increased or decreased in individuals diagnosed with Alzheimer's disease. Accordingly, these biomarkers may be used to assess cognitive function, and/or to diagnose and/or aid in the diagnosis of Alzheimer's disease, and/or to measure progression of Alzheimer's disease in individuals.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook, et al., 2001) Cold Spring Harbor Press; Oligonucleotide Synthesis M. J. Gait, ed. (1984); Methods in Molecular Biology, Humana Press; Cell Biology: A Laboratory Notebook J. E. Cellis, ed. (1998) Academic Press; Animal Cell Culture R. I. Freshney, ed. (1987); Introduction to Cell and Tissue Culture J. P. Mather and P. E. Roberts (1998) Plenum Press; Cell and Tissue Culture: Laboratory Procedures A. Doyle, J. B. Griffiths, and D. G. Newell, eds. (1993-98) J. Wiley and Sons; Methods in Enzymology Academic Press, Inc.; Handbook of Experimental Immunology D. M. Weir and C. C. Blackwell, eds.; Gene Transfer Vectors for Mammalian Cells J. M. Miller and M. P. Calos, eds., (1987); Current Protocols in Molecular Biology F. M. Ausubel, et al. eds. (1987); PCR: The Polymerase Chain Reaction Mullis, et al eds. (1994); Current Protocols in Immunology J. E. Coligan et al. eds. (1991); Short Protocols in Molecular Biology Wiley and Sons (1999); Immunobiology C. A. Janeway and P. Travers (1997); Antibodies P. Finch (1997); Antibodies: A Practical Approach D. Catty., ed. (1988-1989) IRL Press; Monoclonal Antibodies: A Practical Approach P. Shepherd and C. Dean, eds. (2000) Oxford University Press; Using Antibodies: A Laboratory Manual E. Harlow and D. Lane (1999) Cold Spring Harbor Laboratory Press; The Antibodies M. Zanetti and J. D. Capra, eds. (1995) Harwood Academic Publishers.

### DEFINITIONS

As used herein, the terms “Alzheimer's patient”, “AD patient”, and “individual diagnosed with AD” all refer to an individual who has been diagnosed with AD or has been given a probable diagnosis of Alzheimer's disease (AD).

As used herein, methods for “aiding diagnosis” refer to methods that assist in making a clinical determination regarding the presence, nature or stage, of Alzheimer's disease or mild cognitive impairment (MCI), and may or may not be conclusive with respect to the definitive diagnosis. Accordingly, for example, a method of aiding diagnosis of AD can comprise measuring the amount of one or more AD biomarkers in a biological sample from an individual.

As used herein, the phrase “biological fluid sample” encompasses a variety of fluid sample types obtained from an individual. The definition encompasses blood, cerebral spinal fluid (CSF), urine and other liquid samples of biological origin. The definition also includes samples that have been manipulated in any way after their procurement, such as by treatment with reagents, solubilization, or enrichment for certain components, such as proteins or polynucleotides. Biological fluid samples may be used in diagnostic or monitoring assays.

As used herein, the term “peripheral biological fluid sample” refers to a biological fluid sample that is not derived from the central nervous system (i.e., is not a CSF sample) and includes blood samples and other biological fluids not derived from the CNS.

As used herein, the term “blood sample” is a biological sample which is derived from blood, preferably peripheral (or circulating) blood. A blood sample may be, for example, whole blood, plasma or serum.

As used herein, the term “individual” refers to a mammal, more preferably a human. Mammals include, but are not limited to, humans, primates, farm animals, sport animals, rodents and pets.

As used herein, the term “normal” individual or a sample from a “normal” individual or “control” individual for quantitative and qualitative data refers to an individual who has or would be assessed by a physician as not having AD, MCI, or other memory deficiency disorders and has an Mini-Mental State Examination (MMSE) score or would achieve a MMSE score in the range of 25-30 (MMSE referenced in Folstein et al. (1975) J. Psychiatr. Res. 12:1289-198). A “normal” individual is generally age-matched within a range of 5 to 10 years, including but not limited to, an individual that is age-matched, with the individual to be assessed.

As used herein, the phrase “individual with mild AD” refers to an individual who (a) has been diagnosed with AD or has been given a diagnosis of probable AD, and (b) has either been assessed with the MMSE and scored 22-27 or would achieve a score of 22-27 upon MMSE testing. Accordingly, “mild AD” refers to AD in a individual who has either been assessed with the MMSE and scored 22-27 or would achieve a score of 22-27 upon MMSE testing.

As used herein, the phrase “individual with moderate AD” refers to an individual who (a) has been diagnosed with AD or has been given a diagnosis of probable AD, and (b) has either been assessed with the MMSE and scored 16-21 or would achieve a score of 16-21 upon MMSE testing. Accordingly, “moderate AD” refers to AD in a individual who has either been assessed with the MMSE and scored 16-21 or would achieve a score of 16-21 upon MMSE testing.

As used herein, the phrase “individual with severe AD” refers to an individual who (a) has been diagnosed with AD or has been given a diagnosis of probable AD, and (b) has either been assessed with the MMSE and scored 12-15 or would achieve a score of 12-15 upon MMSE testing. Accordingly, “severe AD” refers to AD in a individual who has either been assessed with the MMSE and scored 12-15 or would achieve a score of 12-15 upon MMSE testing. Individuals with severe AD may score lower than 12 upon MMSE testing.

As used herein, the term “treatment” or “treating” refers to any of the alleviation, amelioration, and/or stabilization of a symptom, as well as delay in progression of a symptom of a particular disorder. For example, “treatment” of AD includes any one or more of: amelioration and/or elimination of one or more symptoms of AD, reduction of one or more symptoms of AD, stabilization of the symptoms of AD (e.g., failure to progress to more advanced stages of AD), and delay in progression (i.e., worsening) of one or more symptoms of AD.

As used herein, the phrase “delaying the development” or “delaying the progression” of AD means to defer, hinder, slow, retard, stabilize and/or postpone the progression of any aspect of AD, such as cognitive function impairment. This delay can be of varying lengths of time, depending on the history of the disease and/or individual being treated. A method that “delays” development of the symptoms is a method that reduces probability of developing the symptom in a given time frame and/or reduces the extent of one or more symptoms in a given time frame, when compared to not using the method. Such comparisons are typically based on clinical studies, using a statistically significant number of individuals.

As used herein, the term “prevention” or “preventing” refers to any of: halting the onset of AD, reducing the risk of development of AD, reducing the incidence of AD, delaying the onset of AD, reducing the development of symptoms of AD, delaying the onset of symptoms of AD, increasing the time to onset of symptoms of AD.

As used herein, the term “effective amount” or “therapeutically effective amount” refers to an amount sufficient to effect beneficial or desired clinical results including, but not limited to, any one or more of ameliorating a symptom of AD, (such as improving any accompanying cognitive and/or memory deficit(s) which are characteristic of AD), or delaying the development of AD or delaying onset of AD or decreasing the risk of development of AD.

As used herein, the phrase “fold difference” refers to a numerical representation of the magnitude difference between a measured value and a reference value for an AD-associated biomarker. Fold difference is calculated mathematically by division of the numeric measured value with the numeric reference value. For example, if a measured value for an AD-associated biomarker is 20 nanograms/milliliter (ng/ml), and the reference value is 10 ng/ml, the fold difference is 2 (20/10=2) or 2-fold increase (a 100% increase). Alternatively, if a measured value for an AD biomarker is 10 nanograms/milliliter (ng/ml), and the reference value is 20 ng/ml, the fold difference is 0.5 (10/20=0.50) or −50% (a 50% reduction).

As used herein, the terms “polypeptide”, “polypeptide factor” or “protein” encompasses full length molecules as well as fragments thereof that retain at least one biological activity.

As used herein, “a”, “an”, and “the” can mean singular or plural (i.e., can mean one or more) unless indicated otherwise.

With respect to all methods described herein, references to a polypeptide factor (product), an agonist, or an antagonist, also include compositions comprising one or more of these agents. These compositions may further comprise suitable excipients, such as pharmaceutically acceptable excipients (carriers) including buffers, which are well known in the art. The present invention can be used alone or in combination with other conventional methods of treatment.

### Patient Population

One of skill in the art, i.e., a skilled physician, diagnoses Alzheimer's disease (AD) using a series of examinations and tests. No single test can detect Alzheimer's disease, instead diagnosis usually involves a thorough medical history and physical examination (including a neurological exam) as well as tests to assess memory and the overall function of the mind and nervous system. While memory impairment is a necessary feature for the diagnosis of Alzheimer's disease, changes in other areas must be present also. These other areas include, but are not limited to, language, decision-making ability, judgment, attention and personality.

Symptoms of Alzheimer's disease include, but are not limited to, memory impairment, memory loss, difficulty performing familiar tasks, problems with language, disorientation to time and place, poor or decreased judgment, problems with abstract thinking, misplacing things, changes in mood or behavior, changes in personality and loss of initiative. In early stages, the symptoms of AD may be subtle and resemble signs that people mistakenly attribute to “natural aging”. In moderate to more advanced stages, the symptoms become more obvious and severe cases or the end stages of AD, a person can no longer survive without assistance. As described herein, decreased levels of at least one AD-associated biomarker selected from IL-1α, PDGF-BB, TNF-α, M-CSF, G-CSF, GDNF, eotaxin 2, and MCP-3 may be an indication of a symptom or symptoms of AD. Increased levels of at least one AD-associated biomarker selected from PARC, AgRP, MSP-α and BTC may be an indication of a symptom or symptoms of AD.

There are several generally recognized risk factors for Alzheimer's disease and several others that are still being assessed. The well-documented risks include: advanced age, a positive family history of the disease and genetics (heredity). Other risk factors include gender (more women are affected than men), a previous significant head injury, Down syndrome, environmental toxins, low education level and decreases in hormone levels. (See www.alz.org; www.alzherimersdisease.com.) Individuals with one or more risk factor(s) for developing Alzheimer's disease may be administered treatment to prevent the onset of Alzheimer's disease, reduce the risk of developing Alzheimer's disease, reduce the incidence of Alzheimer's disease, and/or delay the onset of Alzheimer's disease.

Mild cognitive impairment (MCI) is a general term most commonly used to describe a subtle but measurable memory disorder. A person with MCI has memory problems greater than normally expected with aging but does not show other symptoms of dementia, such as impaired judgment or reasoning. One set of practice guidelines identified the following criteria for a MCI diagnosis: an individual's report of his or her own memory problems, measurable, greater-than-normal memory impairment detected with standard assessment tests, but normal overall thinking and reasoning skills and the ability to perform daily activities. Although there is not total agreement, MCI may be considered to be a prodromal stage of Alzheimer's disease and depending on the stringency of inclusion criteria, 16-41% of MCI cases progress to Alzheimer's disease each year (Gauthier S. (2006) Lancet 367:1262-70). Individuals diagnosed with MCI may be administered treatment to prevent the onset of Alzheimer's disease, reduce the risk of developing Alzheimer's disease, reduce the incidence of Alzheimer's disease, and/or delay the onset of Alzheimer's disease.

During evaluation to determine which thinking and memory functions may be affected and to what degree, an individual is asked questions to measure cognitive functions for attention, learning, recall, language and visuospatial abilities. The tests are compared to the tests of other individuals of similar age and education. The Mini Mental State Examination (MMSE) is the most commonly used test for complaints of memory problems or when a diagnosis of dementia or Alzheimer's disease is being considered. The MMSE is a tool that can be used to systematically and thoroughly assess mental status. It is a series of questions and tests that measure five areas of cognitive function, i) orientation, ii) registration (memory part 1), iii) attention and calculation, iv) recall (memory part 2), and v) language (which includes writing and drawing). If every answer is correct, a maximum score of 30 points is possible. People with Alzheimer's disease generally score 26 points or less.

Alzheimer's disease (AD) can be classified into stages including probable, mild, moderate and severe. A “probable AD” diagnosis may be given when other causes of symptoms, such as Parkinson's disease, strokes, tumors, etc., have been ruled out. “Mild AD” is associated with a MMSE score of about 22-27; “moderate AD” is associated with a MMSE score of about 16-21 and “severe AD” is associated with a MMSE score of about 12-15. MMSE scores from patients with severe AD may be lower than 12. It should be understood that the MMSE is not a specific test for Alzheimer's disease and that one of skill in the art views MMSE scores in the context of other diagnostic results for a diagnosis of Alzheimer's disease.

### Identification of Biomarkers

To identify changes in protein expression levels or in patterns of expressed proteins characteristic of Alzheimer's disease, the protein levels of 120 cytokines, chemokines, growth factors, soluble receptors and hormone-like proteins were determined. Using antibody-based, arrayed sandwich ELISAs, biological fluid samples obtained from individuals diagnosed with Alzheimer's disease and from control (non-AD) individuals were analyzed as described herein. Biological fluid samples included peripheral biological fluid samples, blood, plasma and serum.

Plasma samples were assayed using a sandwich-format ELISA on a nitrocellulose filter substrate. An antibody array was immobilized on the nitrocellulose filter; a sample was added to the filter and incubated; after removing unbound sample, a mixture of labeled soluble “capture” antibodies was added to the filter; after removing unbound capture antibodies, a detection system (i.e., chemiluminescence) was used to detect the bound, labeled antibodies; the signals associated with the bound antibodies were quantified. Expression data from each sample were normalized to the median expression of all 120 proteins and then scaled using Fisher's Z-transformation for statistical analysis.

To determine whether a subset of the 120 proteins could be used to characterize and classify an unknown plasma sample, a shrunken centroid algorithm packaged in the “predictive analysis of microarray” (PAM) statistical tool was used. (See R. Tibshirani et al. (2002) PNAS 99:6567-6572, specifically incorporated herein by reference.) PAM is a data mining method used for analyzing gene or protein microarray data. It classifies samples of gene or protein expression data into specific groups. PAM performs a training routine followed by cross-validation of identified markers to come up with a ranking for markers that best discriminates between two or more sample sets.

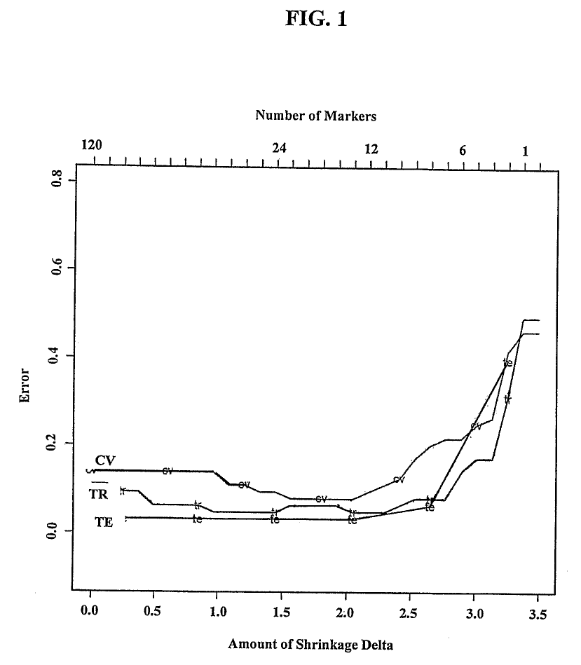

Samples were analyzed using this statistical algorithm to identify a minimal set of biomarkers that can discriminate and predict a class, such as predictive AD-associated biomarkers. To use PAM, the protein expression results from 98 biological samples, 48 AD samples and 50 non-demented controls (NDC), were compiled. From this set of 98, 65 samples were selected to be used as a “training set” in the PAM training routine. AD and NDC samples were randomly divided into two groups that contained equal numbers of AD and NDC samples. The training set was used to train the algorithm to find and validate discriminatory markers. 1 to 120 biomarkers were used to determine the number of predictor biomarkers and their error rate in predicting class for the samples. To determine if the predictors identified by the training routine were over-fitting, 10-fold cross-validation of the samples in the training set was performed. 60 of the training samples were randomly divided into 10 equal-size parts (6 samples/part). The 10 parts were roughly balanced, ensuring that the classes (AD vs NDC) were distributed proportionally among each of the 10 parts. Ten-fold cross-validation works as follows: 90% of the sample were fit to the model and the class labels were predicted of the remaining 10% (test samples). This procedure was repeated 10 times, with each part playing the role of the 10% test samples at least one time and the errors of all 10 parts were added together to compute the overall error. Out of the 120 proteins, PAM identified and confirmed in cross-validation that as few as 12 markers were sufficient to classify the AD and NDC samples. These twelve were identified as the smallest number of biomarkers which had the lowest error rate for predicting class in the training routine and the 10-fold cross-validation and were identified as predictive biomarkers. Training set data, cross-validation data and test set data from PAM analysis of 1-120 biomarkers are shown in FIG. 1.

From the original set of 98 samples, the remaining 33 samples were used as a “test set” in an unbiased independent test. The 33 samples included 16 AD samples and 17 control samples. This test was to determine the accuracy of the twelve biomarkers identified and selected in the training routine and cross-validation in predicting classes of samples. Using the 12 identified biomarkers, PAM was able to identify 100% (16/16) of the AD cases and 94% (16/17) of the NDC controls demonstrating that the relative plasma levels of these 12 biomarkers were characteristic of these two groups.

### Biomarkers

The twelve biomarkers identified in the PAM analysis were interleukin 1α (IL-1α), platelet-derived growth factor beta chain (PDGF-BB), tumor necrosis factor-α (TNF-α), macrophage colony stimulating factor (M-CSF), granulocyte colony-stimulating factor (G-CSF), glial cell line-derived neurotrophic factor (GDNF), eotaxin 2, monocyte chemotactic protein 3 (MCP-3), pulmonary and activation-regulated chemoline (PARC), agouti-related protein (AgRP), macrophage stimulating protein-α (MSP-α) and betacellulin (BTC) and are listed in Table 1. The 12 identified biomarkers appeared to be predictive across all stages of AD including mild, moderate and severe. Stages of AD as determined by for example, MMSE scores, are known in the art.

TABLE 1

| AD- | | | | | |
|---|---|---|---|---|---|
| Associated | | | q-value | Localfdr | SEQ ID |
| Biomarker | Score(d) | Fold-change | (%) | (%) | No. |
| IL-1α | −4.953750235 | 0.59944766 | 0 | 2.2241109 | 1 |
| PDGF-BB | −5.183828516 | 0.23789468 | 0 | 1.3683509 | 2 |
| TNF-α | −5.331956877 | 0.49429852 | 0 | 0.8140407 | 3 |
| M-CSF | −4.858727026 | 0.55991324 | 0 | 2.5706375 | 4 |
| G-CSF | −4.457906727 | 0.57886252 | 0 | 3.8783287 | 5 |
| GDNF | −4820153765 | 0.60845020 | 0 | 2.7088013 | 6 |
| Eotaxin 2 | −3.684471682 | 0.61096904 | 0 | 5.3285777 | 7 |
| MCP-3 | −3.365524849 | 0.68535847 | 0 | 5.9512204 | 8 |
| PARC | 4.435887079 | 2.12300997 | 0 | 0.0468847 | 9 |
| AgRP | 3.874304406 | 1.89358090 | 0 | 0.1291116 | 10 |
| MSP-α | 2.808370992 | 1.63150081 | 0 | 1.2308643 | 11 |
| BTC | 4.137732669 | 1.50719061 | 0 | 0.0974223 | 12 |

One subset of these 12 biomarkers was observed to be at decreased levels in the biological sample(s) (plasma) of individuals diagnosed with Alzheimer's disease as compared with controls (that is, samples from control individuals) and included IL-1α, PDGF-BB, TNF-α, M-CSF, G-CSF, GDNF, eotaxin 2, and MCP-3 (listed in Table 2).

TABLE 2

| AD- | | | | | |
|---|---|---|---|---|---|
| Associated | | | q-value | Localfdr | SEQ ID |
| Biomarker | Score(d) | Fold-change | (%) | (%) | No. |
| IL-1α | −4.953750235 | 0.59944766 | 0 | 2.2241109 | 1 |
| PDGF-BB | −5.183828516 | 0.23789468 | 0 | 1.3683509 | 2 |
| TNF-α | −5.331956877 | 0.49429852 | 0 | 0.8140407 | 3 |
| M-CSF | −4.858727026 | 0.55991324 | 0 | 2.5706375 | 4 |
| G-CSF | −4.457906727 | 0.57886252 | 0 | 3.8783287 | 5 |
| GDNF | −4820153765 | 0.60845020 | 0 | 2.7088013 | 6 |
| Eotaxin 2 | −3.684471682 | 0.61096904 | 0 | 5.3285777 | 7 |
| MCP-3 | −3.365524849 | 0.68535847 | 0 | 5.9512204 | 8 |

Another subset of these 12 biomarkers was observed to be at increased levels in the biological samples (plasma) of individuals diagnosed with Alzheimer's disease as compared with controls and included PARC, AgRP, MSP-α and BTC (listed in Table 3).

TABLE 3

| AD- | | | | | |
|---|---|---|---|---|---|
| Associated | | | q-value | Localfdr | SEQ ID |
| Biomarker | Score(d) | Fold-change | (%) | (%) | No. |
| PARC | 4.435887079 | 2.12300997 | 0 | 0.0468847 | 9 |
| AgRP | 3.874304406 | 1.89358090 | 0 | 0.1291116 | 10 |
| MSP-α | 2.808370992 | 1.63150081 | 0 | 1.2308643 | 11 |
| BTC | 4.137732669 | 1.50719061 | 0 | 0.0974223 | 12 |

Table 4 provides the amino acid sequences of the identified 12 biomarkers.

TABLE 4

| | | |
|---|---|---|
| SEQ | | |
| ID NO | Biomarker | Amino Acid Sequence |

| | | |
|---|---|---|
| 1 | IL-1α | MAKVPDMFED LKNCYSENEE DSSSTDHLSL NQKSFYHVSY |
| | | GPLHEGCMDQ SVSLSISETS KTSKLTFKES MVVVATNGKV |
| | | LKKRRLSLSQ SITDDDLEAI ANDSEEEIIK PRSAPFSFLS |
| | | NVKYNFMRII KYEFILNDAL NQSIIRANDQ YLTAAALHNL |
| | | DEAVKFDMGA YKSSKDDAKI TVILRISKTQ LYVTAQDEDQ |
| | | PVLLKEMPEI PKTITGSETN LLFFWETHGT KNYFTSVAHP |
| | | NLFIATKQDY WVCLAGGPPS ITDFQILENQ A |
| 2 | PDGF-BB | MNRCWALFLS LCCYLRLVSA EGDPTPEELY EMLSDHSIRS |
| | | FDDLQRLLHG DPGEEDGAEL DLNMTRSHSG GELESLARGR |
| | | RSLGSLTIAE PAMIAECKTR TEVFEISRRL IDRTNANFLV |
| | | WPPCVEVQRC SGCCNNRNVQ CRPTQVQLRP VQVRKIEIVR |
| | | KKPIFKKATV TLEDHLACKC ETVAAARPVT RSPGGSQEQR |
| | | AKTPQTRVTI RTVRVRRPPK GKHRKFKHTH DKTALKETLG |
| | | A |
| 3 | TNF-α | MSTESMIRDV ELAEEALPKK TGGPQGSRRC LFLSLFSFLI |
| | | VAGATTLFCL LHFGVIGPQR EEFPRDLSLI SPLAQAVRSS |
| | | SRTPSDKPVA HVVANPQAEG QLQWLNRRAN ALLANGVELR |
| | | DNQLVVPSEG LYLIYSQVLF KGQGCPSTHV LLTHTISRIA |
| | | VSYQTKVNLL SAIKSPCQRE TPEGAEAKPW YEPIYLGGVF |
| | | QLEKGDRLSA EINRPDYLDF AESGQVYFGI IAL |
| 4 | M-CSF | MTAPGAAGRC PPTTWLGSLL LLVCLLASRS ITEEVSEYCS |
| | | HMIGSGHLQS LQRLIDSQME TSCQITFEFV DQEQLKDPVC |
| | | YLKKAFLLVQ DIMEDTMRFR DNTPNAIAIV QLQELSLRLK |
| | | SCFTKDYEEH DKACVRTFYE TPLQLLEKVK NVFNETKNLL |
| | | DKDWNIFSKN CNNSFAECSS QDVVTKPDCN CLYPKAIPSS |
| | | DPASVSPHQP LAPSMAPVAG LTWEDSEGTE GSSLLPGEQP |
| | | LHTVDPGSAK QRPPRSTCQS FEPPETPVVK DSTIGGSPQP |
| | | RPSVGAFNPG MEDILDSAMG TNWVPEEASG BASEIPVPQG |
| | | TELSPSRPGG GSMQTEPARP SNFLSASSPL PASAKGQQPA |
| | | DVTGTALPRV GPVRPTGQDW NHTPQKTDHP SALLRDPPEP |
| | | GSPRISSLRP QGLSNPSTLS AQPQLSRSHS SGSVLPLGEL |
| | | EGRRSTRDRR SPAEPEGGPA SEGAARPLPR FNSVPLTDTG |
| | | HERQSEGSSS PQLQESVFHL LVPSVILVLL AVGGLLFYRW |
| | | RRRSHQEPQR ADSPLEQPEG SPLTQDDRQV ELPV |
| 5 | G-CSF | MAGPATQSPM KLMALQLLLW HSALWTVQEA TPLGPASSLP |
| | | QSFLLKCLEQ VRKIQGDGAA LQEKLVSECA TYKLCHPEEL |
| | | VLLGHSLGIP WAPLSSCPSQ ALQLAGCLSQ LHSGLFLYQG |
| | | LLQALEGISP ELGPTLDTLQ LDVADFATTI WQQMEELGMA |
| | | PALQPTQGAM PAFASAFQRR AGGVLVASHL QSFLEVSYRV |
| | | LRHLAQP |
| 6 | GDNF | MKLWDVVAVC LVLLHTASAF PLPAGKRPPE APAEDRSLGR |
| | | RRAPFALSSD SNMPEDYPDQ FDDVMDFIQA TIKRLKRSPD |
| | | KQMAVLPRRE RNRQAAAANP ENSRGKGRRG QRGKNRGCVL |
| | | TAIHLNVTDL GLGYETKEEL IFRYCSGSCD AAETTYDKIL |
| | | KNLSRNRRLV SDKVGQACCR PIAFDDDLSF LDDNLVYHIL |
| | | RKHSAKRCGC I |
| 7 | Eotaxin2 | MAGLMTIVTS LLFLGVCAHH IIPTGSVVIP SPCCMFFVSK |
| | | RIPENRVVSY QLSSRSTCLK AGVIFTTKKG QQFCGDPKQE |
| | | WVQRYMKNLD AKQKKASPRA RAVAVKGPVQ RYPGNQTTC |
| 8 | MCP-3 | MKASAALLCL LLTAAAFSPQ GLAQPVGINT STTCCYRFIN |
| | | KKIPKQRLES YRRTTSSHCP REAVIFKTKL DKEICADPTQ |
| | | KWVQDFMKHL DKKTQTPKL |
| 9 | PARC | MKGLAAALLV LVCTMALCSC AQVGTNKELC CLVYTSWQIP |
| | | QKFIVDYSET SPQCPKPGVT LLTKRGRQIC ADPNKKWVQK |
| | | YISDLKLNA |
| 10 | AgRP | MLTAAVLSCA LLLALPATRG AQMGLAPMEG IRRPDQALLP |
| | | ELPGLGLRAP LKKTTAEQAE EDLLQEAQAL AEVLDLQDRE |
| | | PRSSRRCVRL HESCLGQQVP CCDPCATCYC RFFNAFCYCR |
| | | KLGTAMNPCS RT |
| 11 | MSP-3 | MGWLPLLLLL TQYLGVPGQR SPLNDFQVLR GTELQHLLHA |
| | | VVPGPWQEDV ADAEECAGRC GPLMDCRAFH YNVSSHGCQL |
| | | LPWTQHSPHT RLRRSGRCDL FQKKDYVRTC IMNNGVGYRG |
| | | TMATTVGGLP CQAWSHKFPN DHKYTPTLRN GLEENFCRNP |
| | | DGDPGGPWCY TTDPAVRFQS CGIKSCREAA CVWCNGEEYR |
| | | GAVDRTESGR ECQRWDLQHP HQHPFEPGKF LDQGLDDNYC |
| | | RNPDGSERPW CYTTDPQIER EFCDLPRCGS EAQPRQEATT |
| | | VSCFRGKGEG YRGTANTTTA GVPCQRWDAQ IPHQHRFTPE |
| | | KYACKDLREN FCRNPDGSEA PWCFTLRPGM RAAFCYQIRR |
| | | CTDDVRPQDC YHGAGEQYRG TVSKTRKGVQ CQRWSAETPH |
| | | KPQFTFTSEP HAQLEENFCR NPDGDSHGPW CYTMDPRTPF |
| | | DYCALRRCAD DQPPSILDPP DQVQFEKCGK RVDRLDQRRS |
| | | KLRVVGGHPG NSPWTVSLRN RQGQHFCGGS LVKEQWILTA |
| | | RQCFSSCHMP LTGYEVWLGT LFQNPQHGEP SLQRVPVAKM |
| | | VCGPSGSQLV LLKLERSVTL NQRVALICLP PEWYVVPPGT |
| | | KCEIAGWGET KGTGNDTVLN VALLNVISNQ ECNIKHRGRV |
| | | RESEMCTEGL LAPVGACEGD YGGPLACFTH NCWVLEGIII |
| | | PNRVCARSRW PAVFTRVSVF VDWIHKVMRL G |
| 12 | BTC | MDRAARCSGA SSLPLLLALA LGLVILHCVV ADGNSTRSPE |
| | | TNGLLCGDPE ENCAATTTQS KRKGHFSRCP KQYKHYCIKC |
| | | RCRFVVAEQT PSCVCDEGYI GARCERVDLF YLRCDRGQIL |
| | | VICLIAVMVV FIILVIGVCT CCHPLRKRRK RKKKEEEMET |
| | | LGKDITPINE DIEETNIA |

The twelve biomarkers identified in the PAM analyses included cytokines, chemokines, growth factors, neurotrophic factors, and neuropeptide hormone-like molecules and each is described briefly below.

Interleukin-1α (IL-1α) has a broad spectrum of biological activities and can be synthesized by many different cells, including monocytes, macrophages, microglia, fibroblasts, astrocytes, endothelial cells, and lymphocytes. IL-1α has several alternative names including hematopoietin-1, catabolin, monocyte cell factor (MCF) and leukocyte endogenous factor (LEM). The biological activities of IL-1α include, but are not limited to, stimulating thymocyte proliferation by inducing IL-2 release; B-cell maturation and proliferation; fibroblast growth factor activity; involvement in the inflammatory response; and stimulating the release of prostaglandin and collagenase from synovial cells. Receptors for IL-1α include IL-1R type I and type II and IL-1Ra.

Platelet-derived growth factor (PDGF-BB) is mainly produced in platelets, but can also be synthesized by macrophages, endothelial cells, megakaryocytes, vascular smooth muscle cells and glial cells. The biological activities of PDGF-BB include, but are not limited to, potent mitogen for cells of mesenchymal origin; release by platelets upon wounding and an important role in stimulation of adjacent cells to proliferate and assist in healing the wound. Receptors for PDGF-BB include PDGF receptor α, β, (a molecule structurally related to M-CSF receptor) and are found on cells of mesenchymal origin including glial cells.

Tumor necrosis factor-α (TNF-α) is a cytokine produced primarily by monocytes and macrophages and has been attributed a central role in most, if not all, inflammatory processes. The biological effects of TNF-α include, but are not limited to, cytotoxicity of cells; regulation of inflammatory processes through induction of other cytokines, (for example, IL-1, IL-6, IL-8, macrophage inflammatory protein (MIP)-2, granulocyte-macrophage colony stimulating factor (GM-CSF) and adhesion molecules); lipid metabolism; coagulation; insulin resistance; and endothelial cell function. Receptors include TNFRSF1A and TNFRSF1B and are found on nearly all cell types except erythrocytes and resting T cells.

Macrophage colony-stimulating factor (M-CSF) is produced by a variety of cell types, including macrophages, monocytes, lymphocytes, endothelial cells and fibroblasts. Alternative names for M-CSF include colony stimulating factor 1 (CSF1) and macrophage-specific colony stimulating factor. The biological activities of M-CSF include, but are not limited to, acting as a growth, differentiation and survival factor for bone marrow progenitor cells of the mononuclear phagocyte lineage; stimulating the proliferation and function of mature macrophages via specific receptors on responding cells. Receptors include CSFIR, FMS and CD115 and are found on macrophages and their progenitors and microglia.

Granulocyte colony-stimulating factor (G-CSF) is the major growth factor involved in the production of neutrophilic granulocytes and is produced by a wide range of cell types. The biological effects of G-CSF include, but are not limited to, stimulating the proliferation and differentiation of progenitor cells for granulocytes; acting as a mediator of anti-infective and inflammatory responses; and stimulating neutrophil proliferation and activity. Receptors include CSFR or GCSFR and are found on hematopoietic progenitor cells.

Glial cell line-derived neurotrophic factor (GDNF) is a protein that may be identified in or obtained from glial cells and which exhibits neurotrophic activity. GDNF is produced by the substantia nigra within the midbrain and astrocytes of the basal forebrain. The biological effects of GDNF include, but are not limited to, increasing survival and differentiation of various neurons including during embryogenesis and after injury; mediating synaptic plasticity in the central nervous system and the peripheral nervous system. Receptors include GDNFR-1 and tyrosine kinase RET and are found in the human anterior pituitary gland.

Eotaxin 2 is a chemokine produced by monocytes and T cells, and is preferentially expressed in polarized human secondary lymphoid follicles. The biological effects of eotaxin 2 include, but are not limited to, recruiting and activating mast cells, eosinophils and resting Th2 cells. It has lower chemotactic activity for neutrophils and none for monocytes and activated lymphocytes. The receptor for eotaxin 2 is CCR3 and is found on eosinophils, mast cells and Th2 cells.

Monocyte chemotactic protein-3 (MCP-3) is a potent chemoattractant of monocytes and dendritic cells, T lymphocytes, basophils and eosinophils but not neutrophils. The biological effects of MCP-3 include, but are not limited to, inflammation and macrophage function; a major role in chronic inflammatory diseases. Receptors include CCR1, CCR2 and CCR3 and are found on monocytes, basophils, T cells, astrocytes, microglia and neurons.

Pulmonary and activation-regulated chemokine (PARC) is produced by dendritic cells, alveolar macrophages, esosinophils, keratinocytes, dermal fibroblasts and chrondroctyes. Alternative names include AMAC-1 (alternative activated macrophage associated C-C-chemokine), MIP-4 (macrophage inflammatory protein-4), DC-CK1 (dendritic cell-derived chemokine-1) and CCL18. PARC can be specifically induced in macrophages by cytokines IL-4, IL-13, and IL-10. PARC is a chemotactic factor that attracts lymphocytes but not monocytes or granulocytes. It attracts naive T lymphocytes toward dendritic cells and activated macrophages into lymph nodes; it has chemotactic activity for naive T cells, CD4+ and CD8+ T cells and it may be involved in B cell migration into B cell follicles in lymph nodes. The receptor for this chemokine is not known at this time.

Agouti-related protein (AgRP) is a neuropeptide hormone-like molecule involved in body weight regulation. AgRP is found most abundantly in the adrenal gland, the hypothalamus, and the subthalamic nucleus. AgRP regulates weight homeostasis and may play a role in the regulation of melanocortin receptors on neurons with the hypothalamus and adrenal gland. The receptor for AgRP is MC4R and it is found on melancortinergic neurons in the arcuate nucleus and the adrenal gland.

Macrophage stimulating protein-α (MSP-α) is a cytokine which belongs to the family of plasminogen-related growth factors. Alternative names for MSP-α include macrophage stimulating protein-1, hepatocyte growth factor-like protein (HGFL) and macrophage stimulating protein alpha chain. MSP-α is produced by the liver and kidney and its biological effects include chemotactic activity on peritoneal macrophages, but not on exudate macrophages or blood monocytes; and stimulation of keratinocyte cell lines causing either chemotactic responses or proliferation in cell culture. The receptor for MSP-α is RON tyrosine kinase and it is found on monocytes, macrophages, keratinocytes and ciliated epithelia within the lung.

Betacellulin (BTC) is a growth factor found within the pancreas, small intestine, liver, kidney, heart and lung. It is a member of the EGF family and has been shown to be a potent mitogen for retinal pigment epithelial cells and vascular smooth muscle cells. BTC is activated in a cascade of events that resembles an inflammatory and/or tissue remodeling process leading to ovulation in mammals. The receptor for BTC is ErbB4 (EGF-R) and is found on most cells.

As will be appreciated by one of skill in the art, additional biomarkers, such as brain-derived neurotrophic factor (BDNF), can be used in the methods disclosed herein including methods to diagnose AD and as the basis for methods of treatment. For example, reduced plasma BDNF levels have been observed in individuals diagnosed with early AD. Accordingly, some methods for treatment of an individual diagnosed with AD comprise administration of a polypeptide factor(s) and/or agonist(s) of BDNF that increases the BDNF level or increases BDNF biological activity in the individual.

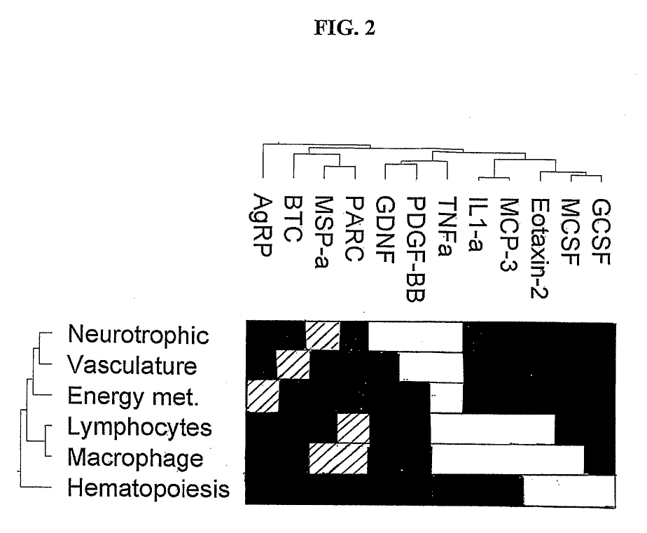

In a functional analysis, the 12 identified biomarkers were shown to be associated with hematopoiesis, energy metabolism, vasculature, immunological function and/or are neurotrophic-associated (FIG. 2). Decreased levels of at least one AD-associated biomarker selected from IL-1α, PDGF-BB, TNF-α, M-CSF, G-CSF, GDNF, eotaxin 2, and MCP-3 may be an indication of a symptom or symptoms of AD. Increased levels of at least one AD-associated biomarker selected from PARC, AgRP, MSP-α and BTC may be an indication of a symptom or symptoms of AD. Accordingly, modulation of these biomarkers, such as for example, IL-1α, PDGF-BB, TNF-α, M-CSF, G-CSF, GDNF, eotaxin-2, MCP-3, PARC, AgRP, MSP-α, and BTC may be useful in the treatment of AD.

Several of the AD-associated biomarkers have been shown to be associated with specific types of cells such as macrophages, monocytes and/or lymphocytes. For example, an additional subset of at least seven biomarkers was observed to be produced by, affected by, or associated with monocytes and/or macrophages and includes IL-1α, TNF-α, M-CSF, eotaxin-2, MCP-3, PARC and MSP-α, and. Differences in levels of these seven biomarkers as compared to control individuals may be evidence of a dysfunction or impairment of the macrophage/monocyte repertoire. Without being bound by theory, a dysfunction or impairment within the macrophage/monocyte repertoire may contribute to, and/or be associated with, the pathology of Alzheimer's disease. Accordingly, modulation of AD-associated biomarkers produced by, and/or affected by, and/or associated with, monocytes and/or macrophages, such as for example, IL-1α, TNF-α, M-CSF, eotaxin-2, MCP-3, PARC and MSP-α, may be useful in the treatment of AD. As used herein, “modulation” means that for biomarkers found to be decreased in individuals diagnosed with AD, methods comprise administration of polypeptide factors and/or agonists of the biomarker and/or agonists of the biomarker receptor that enhance or increase the levels of the biomarker or enhance or increase the biological activity of the biomarker in the individual; for biomarkers found to be increased in individuals diagnosed with AD, methods comprise administration of antagonists of the biomarker or antagonists of the biomarker receptor that decrease the levels of the biomarker or decrease the biological activity of the biomarker in the individual. “Modulation” or “modulating” encompasses a change in an AD-associated biomarker level and changes in the biological activity associated with an AD-associated biomarker including, but not limited to, interaction with a receptor and downstream activities associated with that respective receptor.

### Methods of Treatment

In general, the methods of the invention described herein are based on the identification of AD-associated biomarkers that are either increased or decreased in the plasma of individuals diagnosed with Alzheimer's disease as compared with individuals not diagnosed with AD. It is believed that modulation of these biomarkers can be effective in treating AD, including ameliorating the symptoms of AD, and/or improving the accompanying cognitive and/or memory deficits associated with AD, and/or delaying development of AD, and/or delaying progression of AD and/or preventing AD. Amelioration of the symptoms of AD, improving cognitive and/or memory deficits associated with AD, delaying development of AD, delaying progression of AD or symptoms of AD can be measured by methods known in the art and are described herein.

AD-associated biomarkers including, but not limited to, IL-1α, PDGF-BB, TNF-α, M-CSF, G-CSF, GNDF, MCP-3, and PARC were identified as increased or decreased in the plasma of individuals diagnosed with MCI and/or prior to any diagnosis of Alzheimer's disease using a PAM analysis as described herein. PAM identified 21 of 23 MCI samples as AD wherein the samples were taken 2-4 years before the disease or disease symptoms manifested. These data demonstrated that MCI patients already had levels of biomarkers in their plasma that identified them AD 2-4 years before a clinical diagnosis of AD could be made. (See, Ray et al. (2006) “Early Alzheimer's disease defined by patterns of cellular communication factors in plasma”.) It is believed that AD-associated biomarkers as described herein may be used as diagnostic markers for individuals at risk for developing AD. Accordingly, it is also believed that modulation of AD-associated biomarkers can be effective in preventing AD in individuals who have at least one risk factor for developing AD, have been diagnosed with MCI, have demonstrated a memory deficiency without other AD symptoms and/or in individuals that may be genetically predisposed to developing AD.

Provided herein are methods of treating Alzheimer's disease in an individual (including a mammal, both human and non-human) comprising modulating the biological activity of, or modulating the levels of, any one or more AD-associated biomarkers selected from the group consisting of or alternatively, any one or more of IL-1α, PDGF-BB, TNF-α, M-CSF, G-CSF, GNDF, eotaxin 2, MCP-3, PARC, AgRP, MSP-α, and BTC. In some embodiments, GDNF is modulated in combination with one or more of the biomarkers selected from IL-1α, PDGF-BB, TNF-α, M-CSF, G-CSF, eotaxin 2, MCP-3, PARC, AgRP, MSP-α, and BTC. All the biomarkers identified bind to at least one specific receptor. Accordingly, the present invention encompasses modulation of the biological activity of, or modulating the levels of, any one or more of the cognate receptors for IL-1α, PDGF-BB, TNF-α, M-CSF, G-CSF, GDNF, eotaxin 2, MCP-3, PARC, AgRP, MSP-α, and BTC. Receptor activity may be modulated by, for example, agonists or antagonists of the receptor or its respective downstream signaling molecules in the signaling pathway.

In some embodiments, an AD-associated biomarker is modulated to significantly increase the levels of said AD-associated biomarker in a biological sample. As used herein, “significantly increase” may be an increase of 10%, 20%, 30%, 40%, 50%, 60%, 70%, 80%, 90% or 100%. A significant increase in the level of an AD-associated biomarker may be an increase in level similar or equivalent to a level measured in samples from control individuals who are non-demented and display to symptoms of AD. It should be understood that an increase to “control” or “normal” levels of an AD-associated biomarker may not be necessary for an effective therapeutic effect. As used herein, “control” or “normal” refers to an individual or a sample from an individual who has or would be assessed by a physician as not having AD, MCI, or other memory deficiency disorders.

In some embodiments, an AD-associated biomarker is modulated to significantly decrease the levels of said AD-associated biomarker in a biological sample. As used herein, “significantly decrease” may be an decrease of 10%, 20%, 30%, 40%, 50%, 60%, 70%, 80%, 90% or 100%. A significant decrease in the level of an AD-associated biomarker may be a decrease to level similar or equivalent to a level measured in samples from control individuals who are non-demented and display to symptoms of AD. It should be understood that a decrease to “control” or “normal” levels of an AD-associated biomarker may not be necessary for an effective therapeutic effect.

In some embodiments, the AD-associated biomarkers are decreased in individuals (see Table 2) and treatment comprises administration of a polypeptide factor(s) that has been identified as a biomarker, and/or an agonist(s) of a biomarker that increase the biomarker levels or increase the biological activity associated with the biomarker. Agonists that increase the biological activity associated with an AD-associated biomarker may be agonists that increase the levels of, or the activity of, a receptor for the biomarker. Accordingly, provided herein are methods for treating or preventing Alzheimer's disease in an individual, said method comprising administering a therapeutically effective amount of a) IL-1α, PDGF-BB, TNF-α, M-CSF, G-CSF, GNDF, eotaxin-2 and/or MCP-3; b) a fragment or variant of IL-1α, PDGF-BB, TNF-α, M-CSF, G-CSF, GNDF, eotaxin-2 and/or MCP-3 that retains a biological activity; c) an agonist of IL-1α, PDGF-BB, TNF-α, M-CSF, G-CSF, GNDF, eotaxin-2 and/or MCP-3; d) an agonist of a receptor of GCSF, TNF-α, GDNF, IL-1α, MCP-3, M-CSF, eotaxin 2, and/or PDGF-BB; or e) a combination thereof; to said individual. In some embodiments, the agonist is a small molecule or an antibody. In other embodiments, the agonist is a biomarker mimic or a biomarker structural analog. In other embodiments, the agonist is a nucleic acid that increases expression of at least one biomarker. An “effective amount” is an amount sufficient to effect beneficial or desired clinical results including any one or more of ameliorating a symptom of AD, (such as improving any accompanying cognitive and/or memory deficit(s) which are characteristic of AD), and/or delaying the development of AD and/or preventing the progression of AD and/or preventing AD.

In some embodiments, the AD-associated biomarkers are increased in individuals (see Table 3) and treatment comprises administration of an antagonist of a biomarker that decreases the biomarker levels or decreases the biological activity associated with the biomarker. Antagonists that decrease the biological activity associated with the AD-associated biomarker may be antagonists that decrease the levels of, or the activity of, a receptor for the biomarker. Accordingly, provided herein are methods for treating or preventing Alzheimer's disease in an individual, said method comprising administering a therapeutically effective amount of a) an antagonist of PARC, AgRP, MSP-α and/or BTC; b) an antagonist of a receptor of PARC, AgRP, MSP-α and/or BTC; or c) a combination thereof, to said individual. In some embodiments, the antagonist is a small molecule or antibody. In some embodiments, the antagonist is a biomarker structural analog or a biomarker mimic. In some embodiments, the antagonist is a nucleic acid molecule.

In another aspect, provided herein are methods for ameliorating the symptoms of, and/or delaying the development of, and/or preventing the progression of Alzheimer's disease (or symptoms of AD) in an individual comprising modulating the biological activity of, or modulating the levels of, any one or more AD-associated biomarker(s), wherein the biomarker(s) is selected from the group consisting of IL-1α, PDGF-BB, TNF-α, M-CSF, G-CSF, GNDF, eotaxin 2, MCP-3, PARC, AgRP, MSP-α, and BTC.

In another aspect, provided herein are methods for preventing Alzheimer's disease in an individual comprising modulating the biological activity of, or modulating the levels of, any one or more AD-associated biomarkers selected from the group consisting of IL-1α, PDGF-BB, TNF-α, M-CSF, G-CSF, GNDF, eotaxin 2, MCP-3, PARC, AgRP, MSP-α, and BTC. As used herein, “preventing” encompasses reducing the risk of onset of AD, lowering the incidence of AD, delaying the onset of AD. In some embodiments, prevention comprises the modulation of the biological activity of, or modulating the levels of, any one or more of the cognate receptors for IL-1α, PDGF-BB, TNF-α, M-CSF, G-CSF, GDNF, eotaxin 2, MCP-3, PARC, AgRP, MSP-α, and BTC. Receptor activity may be modulated by, for example, agonists or antagonists of the receptor or its respective downstream signaling molecules in the signaling pathway. In some embodiments, the individual has at least one risk factor for developing AD. In some embodiments, the individual has been diagnosed with MCI. In some embodiments, the individual has a memory deficiency disorder.

In some embodiments, the prevention of AD comprises administration of a polypeptide factor(s) that has been identified as a biomarker, and/or agonists of a biomarker that increase the biomarker levels and/or increases the biological activity associated with the biomarker. Agonists that increase the biological activity associated with a biomarker may be agonists that increase the levels of, or the activity of, a receptor for the biomarker. Accordingly, provided herein are methods for preventing Alzheimer's disease in an individual, said method comprising administering a therapeutically effective amount of a) IL-1α, PDGF-BB, TNF-α, M-CSF, G-CSF, GNDF, eotaxin-2 and/or MCP-3; b) a fragment or variant of IL-1α, PDGF-BB, TNF-α, M-CSF, G-CSF, GNDF, eotaxin-2 and/or MCP-3 that retains a biological activity; c) an agonist of IL-1α, PDGF-BB, TNF-α, M-CSF, G-CSF, GNDF, eotaxin-2 and/or MCP-3; d) an agonist of a receptor of IL-1α, PDGF-BB, TNF-α, M-CSF, G-CSF, GNDF, eotaxin-2 and/or MCP-3; or e) a combination thereof; to said individual.

In some embodiments, the prevention of AD comprises administration of an antagonist of an AD-associated biomarker that decreases the biomarker levels or decreases the biological activity associated with the biomarker. Antagonists that decrease the biological activity associated with the biomarker may be antagonists that decrease the levels of, or the activity of, a receptor for the biomarker. Accordingly, provided herein are methods for preventing Alzheimer's disease in an individual, said method comprising administering a therapeutically effective amount of a) an antagonist of PARC, AgRP, MSP-α and/or BTC; b) an antagonist of a receptor of PARC, AgRP, MSP-α and/or BTC; or c) a combination thereof, to said individual.

### Polypeptide Factors

Provided herein are methods for treating Alzheimer's disease by administering a therapeutically effective amount of a polypeptide factor (which may be a biomarker), such as for example, any one or more of the AD-associated biomarkers listed in Table 2, thereby increasing the level of one or more of the AD-associated biomarkers. The methods may comprise administration of any one or more polypeptide factors including, but not limited to, any biologically active IL-1α polypeptide factor (product), including an IL-1α having the amino acid sequence set forth in SEQ ID NO:1, variants and derivatives thereof that retain at least one biological activity; any biologically active PDGF-BB polypeptide factor, including a PDGF-BB having the amino acid sequence set forth in SEQ ID NO:2, variants and derivatives thereof that retain at least one biological activity; any biologically active TNF-α polypeptide factor, including a TNF-α having the amino acid sequence set forth in SEQ ID NO:3, variants and derivatives thereof that retain at least one biological activity; any biologically active M-CSF polypeptide factor, including a M-CSF having the amino acid sequence set forth in SEQ ID NO:4, variants, and derivatives thereof that retain at least one biological activity; any biologically active G-CSF polypeptide factor, including a G-CSF having the amino acid sequence set forth in SEQ ID NO:5, variants, and derivatives thereof that retain at least one biological activity; any biologically active GDNF polypeptide factor, including a GDNF having the amino acid sequence set forth in SEQ ID NO:6, variants and derivatives thereof that retain at least one biological activity; any biologically active eotaxin 2 polypeptide factor, including an eotaxin 2 having the amino acid sequence set forth in SEQ ID NO:7, variants and derivatives thereof that retain at least one biological activity; and any biologically active MCP-3 polypeptide factor, including a MCP-3 having the amino acid sequence set forth in SEQ ID NO:8, variants, and derivatives thereof that retain at least one biological activity.

The polypeptide factors, variants and derivatives thereof may be isolated or generated by any means known to those skilled in the art. Naturally-occurring polypeptide factors may be isolated from mammalian cell preparations or from mammalian cell lines secreting or expressing the protein product. The polypeptide factors, variants and derivatives thereof may also be chemically synthesized by any means known to those skilled in the art. Polypeptide factors, variants and derivatives thereof may be produced via recombinant techniques since these techniques are known to be capable of producing comparatively high amounts of protein at a high degree of purity. Recombinant polypeptide factors, variants and derivatives thereof may include glycosylated and non-glycosylated forms of the proteins, and may be expressed in bacterial, mammalian, yeast or insect cell systems.

### Agonists

Methods to develop or produce agonists to cellular receptors are well known in the art. For example, several agonist antibodies to receptor molecules have been developed by methods well-known in the art, including, but not limited to, toll-like receptor 4, 4-1BB (CDw 137), β-1 integrin, Fas, TRAIL-R2, CD28, CD40, CD4 and growth hormone receptor. Some agonists have been used to induce a biological activity associated with a receptor, including apoptosis of myeloma cells with an agonist antibody to CD28, apoptosis and tumor regression with an agonist antibody to TRAIL-R2 and activation of lymphocytes by co-stimulation with CD4 antibody.

An “agonist” refers to any molecule that is capable of binding to or combining with a receptor on a cell to mimic or produce a biological activity typical of the naturally occurring substance. Agonist activity can be determined by the degree of stimulation of the receptor, which may be measured by methods known in the art. The term “agonist” implies no specific mechanism of biological action, and is deemed to expressly include and encompass all possible pharmacological, physiological, and biochemical interactions with the receptor and the biological consequences which can be achieved by a variety of different, and chemically divergent, compositions. An “agonist” also refers to any molecule that is capable of increasing a biomarker's protein level or alternatively the biomarker's biological activity. An agonist may increase a biomarker's protein level by inducing increased expression of the biomarker at either the transcriptional or translational level. Exemplary agonists include, but are not limited to, antibodies, compounds or small molecules with the capability to bind to specific receptors and increase or enhance biological activity, biomarker mimics including peptides, biomarker structural analogs, and nucleic acid molecules.

Accordingly, provided herein are methods for the treatment or prevention of Alzheimer's disease comprising modulating the biological activity of any one or more of the AD-associated biomarkers listed in Table 2. In some embodiments, the methods comprise administering a therapeutically effective amount of an agonist(s) to the individual wherein the agonist(s) enhances, such as, for example, increases the level of, or increases the biological activity of, one or more of the biomarkers IL-1α, PDGF-BB, TNF-α, M-CSF, G-CSF, GNDF, eotaxin-2 and/or MCP-3.

### Antibody Agonists

The antibodies useful in the present invention encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, rabbit, human, or any other origin (including chimeric or humanized antibodies). The antibodies may comprise a modified constant region, such as a constant region that is immunologically inert, e.g., does not trigger complement-mediated lysis, or does not stimulate antibody-dependent cell-mediated cytotoxicity (ADCC). ADCC activity can be assessed using methods disclosed in U.S. Pat. No. 5,500,362. The constant region may be modified as described in Eur. J. Immunol. (1999) 29:2613-2624; PCT Application No. PCT/GB99/01441; and UK Patent Application No. 9809951.8.

For purposes of this invention, an antibody agonist specific for any one of the receptor(s) of IL-1α, PDGF-BB, TNF-α, M-CSF, G-CSF, GNDF, eotaxin 2, or MCP-3 reacts with the appropriate receptor in a manner that enhances or increases the biological activity of IL-1α, PDGF-BB, TNF-α, M-CSF, G-CSF, GNDF, eotaxin 2, or MCP-3. In some embodiments the antibody is a human antibody which recognized one or more epitopes on a human IL-1α, PDGF-BB, TNF-α, M-CSF, G-CSF, GNDF, eotaxin 2, or MCP-3 receptor. In other embodiments the antibody is a mouse or rat antibody which recognizes one or more epitopes on a human IL-1α, PDGF-BB, TNF-α, M-CSF, G-CSF, GNDF, eotaxin 2, or MCP-3 receptor.

The binding affinity of an antibody can be about 0.10 to about 0.80 nM, about 0.15 to about 0.75 nM and about 0.18 to about 0.72 nM. In some embodiments, the binding affinity is between about 2 pM and 22 pM. In other embodiments, the binding affinity is about 10 nM. In other embodiments, the binding affinity is less than about 10 nM. In other embodiments, the binding affinity is about 0.1 nM or about 0.07 nM. In other embodiments, the binding affinity is less than about 0.1 nM, or less than about 0.07 nM. In other embodiments, the binding affinity is any of about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM.

One way of determining binding affinity of an antibody is by measuring binding affinity of monofunctional Fab fragments of the antibody. To obtain monofunctional Fab fragments, an antibody (for example, IgG) can be cleaved with papain or expressed recombinantly. The affinity of a Fab fragment of an antibody can be determined by surface plasmon resonance (BIAcore3000™ surface plasmon resonance (SPR) system, BIAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the manufacturer's instructions. The polypeptide factor or immunogen of interest can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. The chip can be blocked with ethanolamine. Regeneration studies have shown that a mixture of Pierce elution buffer (Product No. 21004, Pierce Biotechnology, Rockford Ill.) and 4 M NaCl (2:1) effectively removes the bound Fab while keeping the activity of immunogen on the chip for over 200 injections. HBS-EP buffer (0.01M HEPES, pH 7.4, 0.15 NaCl, 3 mM EDTA, 0.005% Surfactant P29) is used as running buffer for the BIAcore assays. Serial dilutions (0.1-10× estimated K_{D}) of purified Fab samples are injected for 1 min at 100 μl/min and dissociation times of up to 2 hours are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R., Roos, H. Fagerstam, L. Petersson, B. (1994) Methods Enzymology 6:99-110) using the BIAevaluation program. Equilibrium dissociation constant (K_{D}) values are calculated as k_{off}/kₒₙ.

Antibodies (e.g, human, humanized, mouse, chimeric) can be made by using immunogens that express full length or partial sequences of particular AD-associated biomarkers or particular receptors. An immunogen comprising a cell that over-expresses an AD-associated biomarker or a particular receptor may be used. An additional immunogen that can be used is a polypeptide that contains a full-length AD-associated biomarker protein or a portion or fragment thereof or a receptor or a portion thereof.

The antibodies may be made by any method known in the art. The route and schedule of immunization of the host animal are generally in keeping with established and conventional techniques for antibody stimulation and production. General techniques for production of human and mouse antibodies are known in the art and are described herein.

It is contemplated that any mammalian subject including humans or antibody-producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from lymphocytes from an immunized host animal and immortalized myeloma cells using the general somatic cell fusion technique of Kohler, B. and Milstein, C. (1975) Nature 256:495-497 or as modified by Buck, D. W., et al. (1982) In Vitro 18:377-381. Available myeloma lines, including but not limited to, X63-Ag8.653 and those from the Salk Institute (Cell Distribution Center, San Diego, Calif., USA) may be used in the procedure. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unfused parental cells. Any of the media described herein, supplemented with or without serum, can be used for culturing hybridomas that secrete monoclonal antibodies. As an alternative to the cell fusion technique, EBV immortalized B cells may be used to produce monoclonal antibodies. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as a source of antibodies encompass all derivatives, progeny cells of the parent hybridomas that produce monoclonal antibodies specific for the immunogen, or a portion thereof wherein the immunogen comprises an AD-associated biomarker or an AD-associated biomarker receptor.

Hybridomas that produce such antibodies may be grown in vitro or in vivo using known procedures. The monoclonal antibodies may be isolated from the culture media or body fluids (i.e. ascites), by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with an immunogen, or a fragment containing the target amino acid sequence conjugated to a protein that is immunogenic in the species to be immunized, (e.g., keyhole limpet hemocyanin, serum albumin, bovine thyroglobulin, or soybean trypsin inhibitor) using a bifunctional or derivatizing agent, (e.g., maleimidobenzoyl sulfosuccinimide ester (conjugation through cysteine residues), N-hydroxysuccinimide (through lysine residues), glytaradehyde, succinic anhydride, SOCl2, or R1N═C═NR, where R and R1 are different alkyl groups), can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in a vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity and greater efficacy in modulating biological activity. It will be apparent to one of skill in the art that one or more polynucleotide changes can be made to an antibody and still maintain its binding ability to the immunogen.

There are four general steps to humanize a monoclonal antibody. These are i) determining the nucleotide and predicted amino acid sequence of the starting antibody light and heavy variable domains, ii) designing the humanized antibody, i.e., deciding which antibody framework region to use during the humanizing process, iii) the actual humanizing methodologies/techniques and iv) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. (1991) Nature 349:293-299; Lobuglio et al. (1989) PNAS 86:4220-4224; Shaw et al. (1987) J. Immunol. 138:4534-4538; and Brown et al. (1987) Cancer Res. 47:3577-3583. Other references describe rodent CDRs grafted into a human supporting framework region (FR) prior to fusion with an appropriate human antibody constant domain. See, for example, Riechmann et al. (1988) Nature 332:323-327; Verhoeyen et al. (1988) Science 239:1534-1536; and Jones et al. (1986) Nature 321:522-525. Another reference describes rodent CDRs supported by recombinantly veneered rodent framework regions. See, for example, European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g. PCT Application No. PCT/GB99/01441; UK Patent Application No. 9809951.8. Other methods of humanizing antibodies that may also be utilized are disclosed by Daugherty et al (1991) Nucl. Acids Res. 19:2471-2476 and in U.S. Pat. Nos. 6,180,377; 6,054,297; 5,997,867; 5,866,692; 6,210,671; and 6,350,861; and in PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse from Abgenix, Inc. (Fremont, Calif.) and HuMAb-Mouse® and TC Mouse™ from Medarex, Inc. (Princeton, N.J.).

Antibodies may be made recombinantly and expressed using any method known in the art such as antibodies made by phage display technology. See, for example, U.S. Pat. Nos. 5,565,332; 5,580,717; 5,733,743; and 6,265,150; and Winter et al. (1994) Annu. Rev. Immunol. 12:433-455. Alternatively, the phage display technology (McCafferty et al. (1990) Nature 348:552-553) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; (for review see, e.g., Johnson, K. S. and Chiswell, D. J. (1993) Current Opinion in Structural Biology 3:564-571). Several sources of V-gene segments can be used for phage display. Clackson et al. (1991) Nature 352:624-628 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al. (1991) J. Mol. Biol. 222:581-597, or Griffith et al. (1993) EMBO J. 12:725-734. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks et al. (1992) Bio/Technol. 10:779-783). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. (1993) 21:2265-2266. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin.

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed.

Antibodies may be made recombinantly by first isolating the antibodies and antibody-producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters et al. (2001) Vaccine 19:2756; Lonberg, N. and D. Huszar (1995) Int. Rev. Immunol. 13:65; and Pollock et al. (1999) J. Immunol. Methods 231:147. Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for any one of the AD-associated biomarkers in Table 1 or the AD-associated biomarker's receptor(s).

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, (Morrison et al (1984) PNAS 81:6851), or by covalently joining to the immunoglobulin coding sequence all or part of the coding sequence for a non-immunoglobulin polypeptide. In that manner, “chimeric” or “hybrid” antibodies are prepared that have the binding specificity of a monoclonal antibody herein.

Antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane (1999) Using Antibodies, a Laboratory Manual, Cold Spring Harbor Laboratory Press, Cold Spring Harbor, N.Y. In an additional example, epitope mapping can be used to determine the sequence to which an antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an antibody. The epitope to which the antibody binds can be determined in a systematic screening by using overlapping peptides derived from the immunogen's sequence and determining binding by the antibody. According to gene fragment expression assays, the open reading frame encoding an immunogen is fragmented either randomly or by specific genetic constructions and the reactivity of the expressed fragments with the antibody to be tested is determined. The gene fragments may, for example, be produced by PCR and then transcribed and translated into protein in vitro, in the presence of radioactive amino acids. The binding of the antibody to radioactively-labeled fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. Mutagenesis of an antigen binding domain, domain swapping experiments and alanine scanning mutagenesis can be performed to identify residues required, sufficient, and/or necessary for epitope binding. For example, domain swapping experiments can be performed using a mutant of the immunogen in which various fragments of the polypeptide have been replaced (swapped) with sequences from a closely related, but antigenically distinct protein (such as another member of the same protein family). By assessing binding of the antibody to the mutant immunogen, the importance of the particular fragment to antibody binding can be assessed.

Yet another method which can be used to characterize an antibody is to use competition assays with other antibodies known to bind to the same antigen, to determine if the antibody binds to the same epitope as other antibodies. Competition assays are well known to those of skill in the art.

### Other Agonists

Agonists other than antibodies specific for the receptors of IL-1α, PDGF-BB, TNF-α, M-CSF, G-CSF, GNDF, eotaxin 2, or MCP-3 may be used to enhance or increase the biological activity, or increase the levels of, the AD-associated biomarkers. Agonists may include, but are not limited to, compounds or small molecules with the capability to bind to specific receptors and increase or enhance biological activity, biomarker mimics of IL-1α, PDGF-BB, TNF-α, M-CSF, G-CSF, GNDF, eotaxin 2, or MCP-3 including peptides, biomarker structural analogs of IL-1α, PDGF-BB, TNF-α, M-CSF, G-CSF, GNDF, eotaxin 2, or MCP-3, compounds or small molecules with the capability to induce, enhance or increase expression of and/or levels of IL-1α, PDGF-BB, TNF-α, M-CSF, G-CSF, GNDF, eotaxin 2, or MCP-3, nucleic acid molecules with the capability to expression IL-1α, PDGF-BB, TNF-α, M-CSF, G-CSF, GNDF, eotaxin 2, or MCP-3 or nucleic acid molecules with the capability to induce, enhance or increase expression of IL-1α, PDGF-BB, TNF-α, M-CSF, G-CSF, GNDF, eotaxin 2, or MCP-3.

In methods comprising small molecules, a small molecule can have a molecular weight of about any of 100 to 20,000 daltons, 500 to 15,000 daltons, or 1000 to 10,000 daltons. Libraries of small molecules are commercially available or can be produced by one skilled in the art.

In some examples, an agonist or “activating compound” binds to any one of the receptors of IL-1α, PDGF-BB, TNF-α, M-CSF, G-CSF, GNDF, Eotaxin 2, MCP-3 wherein activation or enhancement of a biological activity results. As used herein, an “IL-1α, PDGF-BB, TNF-α, M-CSF, G-CSF, GNDF, eotaxin 2, or MCP-3 activating compound” refers to a compound other than an antibody that binds to the receptors of IL-1α, PDGF-BB, TNF-α, M-CSF, G-CSF, GNDF, eotaxin 2, or MCP-3 that directly or indirectly activates, induces, enhances, or increases IL-1α, PDGF-BB, TNF-α, M-CSF, G-CSF, GNDF, eotaxin 2 or MCP-3 biological activity. An “IL-1α, PDGF-BB, TNF-α, M-CSF, G-CSF, GNDF, eotaxin 2, or MCP-3 activating compound” also refers to a compound that activates, induces, enhances or increases expression and/or protein levels of IL-1α, PDGF-BB, TNF-α, M-CSF, G-CSF, GNDF, eotaxin 2 or MCP-3. An activating compound should exhibit any one or more of the following characteristics: (a) bind to any one of IL-1α, PDGF-BB, TNF-α, M-CSF, G-CSF, GNDF, eotaxin 2, or MCP-3 receptors; (b) increase IL-1α, PDGF-BB, TNF-α, M-CSF, G-CSF, GNDF, eotaxin 2, or MCP-3 biological activity or downstream pathways mediated by IL-1α, PDGF-BB, TNF-α, M-CSF, G-CSF, GNDF, eotaxin 2, or MCP-3 signaling function; (c) induce or increase IL-1α, PDGF-BB, TNF-α, M-CSF, G-CSF, GNDF, eotaxin 2, or MCP-3 receptor activation; (d) increase half-life or stabilization of IL-1α, PDGF-BB, TNF-α, M-CSF, G-CSF, GNDF, eotaxin 2, or MCP-3; (e) induce or increase IL-1α, PDGF-BB, TNF-α, M-CSF, G-CSF, GNDF, eotaxin 2, or MCP-3 synthesis, production or release. One skilled in the art can prepare IL-1α, PDGF-BB, TNF-α, M-CSF, G-CSF, GNDF, eotaxin 2, or MCP-3 activating compounds, including but not limited to, small molecule compounds.

In some examples, an agonist or activating compound binds a receptor of IL-1α, PDGF-BB, TNF-α, M-CSF, G-CSF, GNDF, eotaxin 2, or MCP-3. Exemplary sites of targeting (binding) include, but are not limited to, the portion of the receptor that interacts with IL-1α, PDGF-BB, TNF-α, M-CSF, G-CSF, GNDF, eotaxin 2, or MCP-3.

In some examples, an agonist or activating compound induces, enhances or increases the expression (at the transcriptional or translation level) of IL-1α, PDGF-BB, TNF-α, M-CSF, G-CSF, GNDF, eotaxin 2, or MCP-3. Some agonists may bind to DNA to increase gene expression, some agonists may increase mRNA half-life, some agonists may increase protein translation, some agonists may increase protein half-life.

In some examples, the agonist comprises at least one IL-1α, PDGF-BB, TNF-α, M-CSF, G-CSF, GNDF, eotaxin 2, or MCP-3 structural analog. As used herein, “IL-1α, PDGF-BB, TNF-α, M-CSF, G-CSF, GNDF, eotaxin 2, or MCP-3 structural analogs” in the present invention refer to compounds that have a similar 3-dimensional structure as part of that of IL-1α, PDGF-BB, TNF-α, M-CSF, G-CSF, GNDF, eotaxin 2, or MCP-3 and which bind to an IL-1α, PDGF-BB, TNF-α, M-CSF, G-CSF, GNDF, eotaxin 2, or MCP-3 receptor under physiological conditions in vitro or in vivo, wherein the binding at least partially enhances or duplicates an IL-1α, PDGF-BB, TNF-α, M-CSF, G-CSF, GNDF, eotaxin 2, or MCP-3 biological activity. In one example, the IL-1α, PDGF-BB, TNF-α, M-CSF, G-CSF, GNDF, eotaxin 2, or MCP-3 structural analog binds to the correct respective receptor. Suitable IL-1α, PDGF-BB, TNF-α, M-CSF, G-CSF, GNDF, eotaxin 2, or MCP-3 structural analogs can be produced by recombinant methods well known in the art. Structural analogs can also be designed and synthesized through molecular modeling of receptor binding, for example by the method described in PCT Publication No. WO 98/06048. The IL-1α, PDGF-BB, TNF-α, M-CSF, G-CSF, GNDF, eotaxin 2, or MCP-3 structural analogs can be monomers or dimers/oligomers in any desired combination of the same or different structures to obtain improved affinities and biological effects.

### Antagonists

Methods to develop or produce antagonists to soluble molecules and/or to receptors are well known in the art. For example, several antagonist antibodies to soluble molecules or receptor molecules have been developed, including, but not limited to, antibodies to CD40, TrkA, EGFR, toll-like receptor 2, CXCR4, VEGF-R2, VEGF (Avastin®), HER2 (Herceptin®) and IgE (Xolair®). Several non-antibody antagonists have been developed, including, but not limited to, CXCR4 antagonist peptide T140, small molecule integrin antagonist 4-[2-(3,4,5,6-tetrahydropyrimidin-2-ylamino)ethoxy]-benzoyl-2-(5)-aminoethylsulfonylamino-beta-alanine, small molecule LFA-1 antagonist BIRT 377 and glycoprotein IIb/IIIa receptor nonpeptide antagonist tirofiban.

An “antagonist” refers to any molecule that blocks, suppresses or reduces (including significantly) biological activity typical of a specific biomarker or that biomarker's receptor. This can include downstream pathways mediated by receptor signaling, such as receptor binding and/or elicitation of a cellular response to a particular ligand. The term “antagonist” implies no specific mechanism of biological action, and is deemed to expressly include and encompass all possible pharmacological, physiological, and biochemical interactions with the biomarker or the biomarker's receptor and the consequences which can be achieved by a variety of different, and chemically divergent, compositions. Exemplary antagonists include, but are not limited to, antibodies to PARC, AgRP, MSP-α and BTC; anti-sense molecules directed to PARC, AgRP, MSP-α and BTC (including anti-sense molecules directed to a nucleic acid encoding PARC, AgRP, MSP-α and BTC); anti-sense molecules directed to PARC, AgRP, MSP-α or BTC receptors (including anti-sense molecules directed to nucleic acids encoding PARC, AgRP, MSP-α or BTC receptors); PARC, AgRP, MSP-α and BTC inhibitory compounds; PARC, AgRP, MSP-α and BTC structural analogs; dominant-negative mutants of receptors that bind PARC, AgRP, MSP-α or BTC; PARC, AgRP, MSP-α and BTC receptor-immunoadhesins; and antibodies to PARC, AgRP, MSP-α and BTC receptors. For purposes of the present invention, it will be explicitly understood that the term “antagonist” encompasses all the previously identified terms, titles, and functional states and characteristics whereby PARC, AgRP, MSP-α or BTC themselves, PARC, AgRP, MSP-α and BTC biological activities, or the downstream consequences of the biological activities, are substantially nullified, decreased, or neutralized in any meaningful degree.

Accordingly, provided herein are methods for treatment or prevention of Alzheimer's disease comprising modulating the biological activity of, or the levels of, any one or more of the AD-associated biomarkers PARC, AgRP, MSP-α and BTC. In some examples, the modulation comprises administering a therapeutically effective amount of an antagonist to the individual wherein the antagonist inhibits or decreases the biological activity of any one or more of the AD-associated biomarkers PARC, AgRP, MSP-α and BTC. In some examples, the modulation comprises an antagonist which decreases biomarker protein levels. In some examples, an antagonist (e.g., an antibody) binds (physically interacts with) PARC, AgRP, MSP-α or BTC; binds to a PARC, AgRP, MSP-α or BTC receptor; and/or reduces (impedes and/or blocks) downstream receptor signaling. In some examples, an antagonist binds (physically interacts with) PARC, AgRP, MSP-α or BTC. In some examples, an antagonist binds to a PARC, AgRP, MSP-α or BTC receptor. In some examples, an antagonist reduces (impedes and/or blocks) downstream receptor signaling. In some examples, an antagonist inhibits (reduces) PARC, AgRP, MSP-α or BTC synthesis and/or release. In some examples, an antagonist reduces serum or plasma protein levels of PARC, AgRP, MSP-α or BTC.

### Antagonist Antibodies

For a detailed description of antibodies, epitopes, binding affinities, methods of making and screening see above. In another aspect of the invention, methods are provided wherein an antagonist antibody specific for PARC, AgRP, MSP-α or BTC which reacts with PARC, AgRP, MSP-α or BTC, respectively, is administered in a manner that inhibits the protein and/or downstream pathways mediated by the protein's signaling function. In some examples, the antibody is a human antibody which recognizes one or more epitopes on human PARC, human AgRP, human MSP-α or human BTC. In other examples, the antibody is a mouse or rat antibody which recognizes one or more epitopes on human PARC, human AgRP, human MSP-α or human BTC.

In some examples, methods are provided wherein an antagonist antibody specific for a PARC, AgRP, MSP-α or BTC receptor reacts with a PARC, AgRP, MSP-α or BTC receptor, respectively, in a manner that inhibits or blocks the binding of PARC, AgRP, MSP-α or BTC to its respective receptor thereby reducing or inhibiting the protein's biological activity or effect. In other examples, an antagonist antibody specific for a PARC, AgRP, MSP-α or BTC receptor reacts with a PARC, AgRP, MSP-α or BTC receptor and inhibits or blocks downstream pathways mediated by the protein's signaling function, whether or not binding is inhibited. In some examples, the antibody is a human antibody which recognizes one or more epitopes on the receptor for human PARC, on the receptor for human AgRP, on the receptor for human MSP-α or on the receptor for human BTC. In other examples, the antibody is a mouse or rat antibody which recognizes one or more epitopes on the receptor for human PARC, on the receptor for human AgRP, on the receptor for human MSP-α or on the receptor for human BTC.

### Other Antagonists

Antagonists other than antibodies to PARC, AgRP, MSP-α, BTC or their respective receptors may be used. In some examples, an antagonist comprises at least one PARC, AgRP, MSP-α or BTC inhibitory compound. As used herein, a “PARC, AgRP, MSP-α or BTC inhibitory compound” refers to a compound other than an anti-PARC, anti-AgRP, anti-MSP-α or anti-BTC antibody that directly or indirectly reduces, inhibits, neutralizes, or abolishes PARC, AgRP, MSP-α or BTC biological activity. An inhibitory compound should exhibit any one or more of the following characteristics: (a) bind to PARC, AgRP, MSP-α or BTC; (b) inhibit PARC, AgRP, MSP-α or BTC biological activity or downstream pathways mediated by PARC, AgRP, MSP-α or BTC signaling function; (c) block or decrease PARC, AgRP, MSP-α or BTC receptor activation; (d) increase degradation or clearance of PARC, AgRP, MSP-α or BTC; (e) inhibit (reduce) PARC, AgRP, MSP-α or BTC synthesis, production or release. Additional PARC, AgRP, MSP-α or BTC inhibitory compounds are compounds that are competitive inhibitors of PARC, AgRP, MSP-α or BTC. One skilled in the art can prepare PARC, AgRP, MSP-α or BTC inhibitory compounds.

In some examples, an inhibitory compound binds PARC, AgRP, MSP-α or BTC. Exemplary sites of targeting (binding) include, but are not limited to, the portion of PARC, AgRP, MSP-α or BTC that binds to their respective receptors, and those portions of PARC, AgRP, MSP-α or BTC that are adjacent to the receptor-binding region and which are responsible, in part, for the correct three-dimensional shape of the receptor-binding portion. In some examples, an inhibitory compound binds a PARC, AgRP, MSP-α or BTC receptor and inhibits a PARC, AgRP, MSP-α or BTC biological activity. Exemplary sites of targeting include those portions of each receptor that bind to PARC, AgRP, MSP-α or BTC.

In some examples of the invention, an antagonist comprises at least one antisense molecule capable of blocking or decreasing the expression of a functional PARC, AgRP, MSP-α or BTC. In some examples, an antagonist comprises at least one antisense molecule capable of blocking or decreasing the expression of a functional PARC, AgRP, MSP-α or BTC receptor. Nucleotide sequences of PARC, AgRP, MSP-α, BTC and most of their receptors are known and are readily available from publicly available databases. It is routine to prepare antisense oligonucleotide molecules that will specifically bind PARC, AgRP, MSP-α, or BTC mRNA and not cross-react with other polynucleotides. Exemplary sites of targeting for the antisense molecules include, but are not limited to, the initiation codon, the 5′ regulatory regions, the coding sequence and the 3′ untranslated region. In some examples, the antisense oligonucleotides are about 10 to 100 nucleotides in length, about 15 to 50 nucleotides in length, about 18 to 25 nucleotides in length, or more. The antisense oligonucleotides can comprise backbone modifications such as, for example, phosphorothioate linkages and 2′-O sugar modifications well know in the art.

Alternatively, PARC, AgRP, MSP-α or BTC expression and/or release and/or PARC, AgRP, MSP-α or BTC receptor expression can be decreased using methods that are well-known in the art including gene knockdown, morpholino oligonucleotides, RNAi, snRNA or ribozymes.

In some examples, an antagonist comprises at least one PARC, AgRP, MSP-α or BTC structural analog. “PARC, AgRP, MSP-α or BTC structural analogs” in the present invention refer to compounds that have a similar 3-dimensional structure as part of that of PARC, AgRP, MSP-α or BTC and which bind to a PARC, AgRP, MSP-α or BTC receptor under physiological conditions in vitro or in vivo, wherein the binding at least partially inhibits a PARC, AgRP, MSP-α or BTC biological activity. In some examples, the PARC, AgRP, MSP-α or BTC structural analog binds to the correct respective receptor. Suitable structural analogs can be produced by recombinant methods well known in the art. Structural analogs can also be designed and synthesized through molecular modeling of PARC-receptor binding, AgRP-receptor binding, MSP-α-receptor binding or BTC-receptor binding, for example by the method described in PCT Publication No. WO 98/06048. The PARC, AgRP, MSP-α or BTC structural analogs can be monomers or dimers/oligomers in any desired combination of the same or different structures to obtain improved affinities and biological effects.

In other examples, the invention provides an antagonist comprising at least one dominant-negative mutant of a PARC, AgRP, MSP-α or BTC receptor. One skilled in the art can prepare dominant-negative mutants of, e.g., the MSP-α receptor such that the receptor will bind the MSP-α and, thus, act as a “sink” to capture MSP-α. The dominant-negative mutants, however, will not have the normal bioactivity of the MSP-α receptor upon binding to MSP-α. Dominant negative mutants can be administered in polypeptide form or in the form of an expression vector such that the dominant negative mutant, e.g., mutant MSP-α receptor, is expressed in vivo.

In other examples, an antagonist may comprise at least one receptor immunoadhesin. “Receptor immunoadhesins” as used herein refer to soluble chimeric molecules comprising the extracellular domain of a receptor and an immunoglobulin sequence, which retains the binding specificity of the receptor (substantially retains the binding specificity of the receptor) and is capable of binding to ligand.

It is expected that a number of other categories of AD-associated biomarker and AD-associated biomarker receptor antagonists will be identified if sought by the clinician.

### Identification of Biomarker Agonist and Antagonists

Antibody agonists and other agonists can be identified or characterized using methods known in the art, whereby induction, enhancement, or increase of biomarker biological activity, or biomarker expression and/or protein levels, is detected and/or measured. Following initial identification, the activity of a candidate agonist can be further confirmed and refined by bioassays, known to test the targeted biological activities or presence/levels of the biomarker. Alternatively, bioassays can be used to screen candidates directly.

Antibody antagonists and other antagonists can be identified or characterized using methods known in the art, whereby reduction, amelioration, or neutralization of biomarker biological activity, or biomarker expression and/or protein levels, is detected and/or measured. Following initial identification, the activity of a candidate antagonist can be further confirmed and refined by bioassays, known to test the targeted biological activities. Alternatively, bioassays can be used to screen candidates directly.

### Compositions

The compositions used in the methods of the invention comprise an effective amount of a polypeptide factor (product), an agonist or an antagonist, and, in some examples, further comprise a pharmaceutically acceptable excipient. In some examples, the composition is for use in any of the methods described herein. Examples of such compositions, as well as how to formulate, are also described herein. In one example, the composition comprises a polypeptide factor (product) selected from the group consisting of IL-1α, PDGF-BB, TNF-α, M-CSF, G-CSF, GNDF, eotaxin 2, and MCP-3. In other examples, the composition comprises at least two polypeptide factors selected from the group consisting of IL-1α, PDGF-BB, TNF-α, M-CSF, G-CSF, GNDF, eotaxin 2, and MCP-3. In other examples, the composition comprises at least three polypeptide factors selected from the group consisting of IL-1α, PDGF-BB, TNF-α, M-CSF, G-CSF, GNDF, eotaxin 2, and MCP-3. In other examples, the composition comprises three or more polypeptide factors selected from the group consisting of IL-1α, PDGF-BB, TNF-α, M-CSF, G-CSF, GNDF, eotaxin 2, and MCP-3. In some examples, the composition comprises one or more agonists wherein the agonist is an antibody that induces and/or enhances production of IL-1α, PDGF-BB, TNF-α, M-CSF, G-CSF, GNDF, eotaxin 2 and/or MCP-3. In other examples, the composition comprises one or more agonists wherein the agonist is selected from the group comprising small molecule compounds, biomarker mimics, biomarker structural analogs, and nucleic acid molecules and wherein the agonist induces and/or enhances production of IL-1α, PDGF-BB, TNF-α, M-CSF, G-CSF, GNDF, Eotaxin 2 and/or MCP-3.

In some examples, the composition comprises a PARC, AgRP, MSP-α or BTC antagonist. In some examples, the composition comprises one or more PARC, AgRP, MSP-α or BTC antagonists. In other examples, the composition comprises one or more antagonists selected from any one or more of the following, an antagonist (e.g., an antibody) that binds (physically interacts with) PARC, AgRP, MSP-α or BTC; an antagonist that binds to a PARC, AgRP, MSP-α or BTC receptor; and an antagonist that reduces (inhibits and/or blocks) downstream PARC, AgRP, MSP-α or BTC receptor signaling.

A composition used in the present invention can further comprise pharmaceutically acceptable carriers, excipients, or stabilizers (Remington: The Science and Practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™, Pluronics™ or polyethylene glycol (PEG). Pharmaceutically acceptable excipients are further described herein.

### Administration

The polypeptide factors, agonists or antagonists (collectively referred to as “the agent” or “the agents”) may be administered to an individual via any suitable route. For example, the agents may be administered orally, intravenously, sublingually, subcutaneously, intraarterially, intrasynovially, intravescicular (such as via the bladder), intramuscularly, intranasally, intracardiacly, intrathoracicly, intraperitoneally, intraventricularly, intrathecally, intracerebrally, sublingually, by inhalation, by suppository, and transdermally. Administration of a polypeptide factor, an agonist or an antagonist in accordance with the methods in the present invention can be continuous or intermittent, depending, for example, upon the individual's physiological condition, whether the purpose of the administration is therapeutic or prophylactic, and other factors known to skilled practitioners. The administration of an agent may be essentially continuous over a pre-selected period of time or may be in a series of spaced doses.

Accordingly, in some examples, the agent, is administered to a individual in accordance with known administration methods. These administration methods include, but are not limited to, intravenous (e.g., as a bolus or by continuous infusion over a period of time), intramuscular, intraperitoneal, intracerebrospinal, subcutaneous, intra-articular, intrasynovial, intrathecal, oral, inhalation, intranasal or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the agents can be aerosolized using a fluorocarbon formulation and a metered dose inhaler, or inhaled as a lyophilized and milled powder.

In some examples, the agents are administered orally, for example, in the form of tablets, troches, capsules, elixirs, suspensions, syrups, wafers, lollypops, chewing gum or the like prepared by art recognized procedures. It should be apparent to a person skilled in the art that the examples described herein are not intended to be limiting but to be illustrative of the techniques available.

In some examples, the agents are administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of any one or more of the agent may be used for administration. In some examples, the agent may be administered neat. In some examples, the agent comprises an antibody, and may be in various formulations, including formulations comprising a pharmaceutically acceptable excipient. Pharmaceutically acceptable excipients are known in the art, and are relatively inert substances that facilitate administration of a pharmacologically effective substance. For example, an excipient can give form or consistency, or act as a diluent. Suitable excipients include, but are not limited to, stabilizing agents, wetting and emulsifying agents, salts for varying osmolarity, encapsulating agents, buffers, and skin penetration enhancers. Excipients as well as formulations for parenteral and nonparenteral drug delivery are set forth in Remington: The Science and Practice of Pharmacy (2000) 20th Ed. Mack Publishing.

In some examples, the agents are formulated for administration by injection (e.g., intraperitoneally, intravenously, subcutaneously, intramuscularly, etc.). Accordingly, the agents can be combined with pharmaceutically acceptable vehicles such as saline, Ringer's solution, dextrose solution, and the like.

Regardless of the manner of administration, the specific dose is typically calculated according to body weight or body surface area. Further refinement of the appropriate dosage for treatment involving each of the above mentioned agents and/or formulations is routinely made by those of ordinary skill in the art and is within the realm of task routinely performed by those of ordinary skill in the art. Any particular dosage regimen, i.e., dose, timing and repetition, will depend on the agent, the particular individual and that individual's medical history.

An antibody can be administered using any suitable method, including by injection (e.g., intraperitoneally, intravenously, subcutaneously, intramuscularly, etc.). Antibodies can also be administered via inhalation, as described herein. Generally, for administration of antibodies, an initial candidate dosage can be about 2 mg/kg. A typical daily dosage might range from about any of 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient modulation of AD-associated biomarker biological activity is achieved. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one to four times a week is contemplated.

In general, when it is not an antibody, an agonist or an antagonist may (in some examples) be administered at the rate of about 0.1 to 300 mg/kg of the weight of the patient divided into one to three doses, or as disclosed herein. In some examples, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents be administered (such as the half-life of the agent, and other considerations well known in the art).

A small molecule can be administered using any means known in the art, including inhalation, intraperitoneally, intravenously, intramuscularly, subcutaneously, intrathecally, intraventricularly, orally, enterally, parenterally, intranasally, or dermally. In general, when the agonist or antagonist according to the invention is a small molecule, it will be administered at the rate of 0.1 to 300 mg/kg of the weight of the patient divided into one to three, or more doses. For an adult individual of normal weight, doses ranging from 1 mg to 5 g per dose can be administered.

A protein or expression vector can be administered using any means known in the art, such as intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions containing an antisense polynucleotide, expression vector, or subgenomic polynucleotides can also be used. Receptor-mediated DNA delivery techniques are described in, for example, Findeis et al. (1993) Trends Biotechnol. 11:202; Chiou et al. (1994) Gene Therapeutics: Methods And Applications Of Direct Gene Transfer J. A. Wolff, ed.; Wu et al. (1988) J. Biol. Chem. 263:621; Wu et al. (1994) J. Biol. Chem. 269:542; Zenke et al. (1990) PNAS 87:3655; Wu et al. (1991) J. Biol. Chem. 266:338. Therapeutic compositions containing a polynucleotide are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol. The therapeutic polynucleotides and polypeptides of the present invention can be delivered using gene delivery vehicles. The gene delivery vehicle can be of viral or non-viral origin (see generally, Jolly (1994) Cancer Gene Therapy 1:51; Kimura (1994) Human Gene Therapy 5:845; Connelly (1995) Human Gene Therapy 1:185; and Kaplitt (1994) Nature Genetics 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel (1992) Hum. Gene Ther. 3:147 can also be employed.

Non-viral delivery vehicles and methods can also be employed, including, but not limited to, polycationic condensed DNA linked or unlinked to killed adenovirus alone (see, e.g., Curiel (1992) Hum. Gene Ther. 3:147); ligand-linked DNA (see, e.g., Wu (1989) J. Biol. Chem. 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip (1994) Mol. Cell. Biol. 14:2411, and in Woffendin (1994) PNAS 91:1581.

It is also apparent that an expression vector can be used to direct expression of any of the protein-based PARC, AgRP, MSP-α or BTC antagonists described herein (e.g., antibodies, receptor-immunoadhesin, etc.). Similarly, it should also be apparent that an expression vector can be used to direct expression of any of the protein-based IL-1α, PDGF-BB, TNF-α, M-CSF, G-CSF, GNDF, eotaxin 2, and MCP-3 agonists described herein.

Empirical considerations, such as the half-life of a polypeptide, agonist or antagonist, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on the ongoing results from treatment. Alternatively, sustained continuous release formulations of antibodies or other agents may be appropriate. Various formulations and devices for achieving sustained release are known in the art.

In some examples, more than one agent, such as an antibody, may be present in the composition, formulation, etc. The agents can be the same or different from each other. At least one, at least two, at least three, at least four, at least five different agents can be present. Generally, the agents have complementary activities that do not adversely affect each other.

Therapeutic formulations of the protein products, agonists or antagonists (the agents) used in accordance with the present invention are prepared for storage by mixing an agent(s) having the desired degree of purity with optional pharmaceutically acceptable carriers, excipients or stabilizers (Remington: The Science and Practice of Pharmacy (2000) 20th Ed. Mack Publishing), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™, Pluronics™ or polyethylene glycol (PEG).

In some examples, the polypeptide factor, agonists or antagonists (the agents) are administered in liposomes. Liposomes containing a protein product, an agonist or an antagonist (such as an antibody) are prepared by methods known in the art, such as described in Epstein et al. (1985) PNAS 82:3688; Hwang et al (1980) PNAS 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some examples, the polypeptide factor, agonists or antagonists (the agents) are administered in microcapsules or similar formulations. The polypeptide factor, agonists or antagonists (such as an antibody) may be entrapped in microcapsules prepared, for example, by coacervation techniques or by interfacial polymerization (for example, hydroxymethylcellulose or gelatin-microcapsules and poly-(methylmethacylate) microcapsules, respectively), may be entrapped in colloidal drug delivery systems (for example, liposomes, albumin microspheres, microemulsions, nano-particles and nanocapsules) or may be entrapped in macroemulsions. Such techniques are disclosed in Remington: The Science and Practice of Pharmacy (2000) 20th Ed. Mack Publishing.

Sustained-release preparations may be prepared. Suitable examples of sustained-release preparations include semipermeable matrices of solid hydrophobic polymers containing the protein products, agonists or antagonists (such as an antibody), which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic protein compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span® 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid®, Liposyn®, Infonutrol®, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μμm, and have a pH in the range of 5.5 to 8.0.

In some examples, emulsion compositions can be prepared by mixing a polypeptide factor, an agonist or an antagonist with Intralipid® or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some examples, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Assessment of Treatment Methods

Treatment efficacy can be assessed by methods well-known in the art. The progress of treatment is monitored by conventional techniques and assays including, but not limited to, analysis of serum or plasma levels of AD-associated biomarkers (disclosed herein and known in the art) and/or analysis of cognitive ability using standardized clinical dementia assessment scales such as, for example, the mini-mental state examination (MMSE) as described herein.

Transgenic AD mouse models are also available for assessing AD treatments. Several different strains of transgenic mice have been produced that manifest some of the signs and symptoms of Alzheimer's disease. These include age-related memory and learning impairment, loss of brain cells, deposition of amyloid protein in the brain, and tangles of nerve fibers composed of a protein called tau (the latter two are among the hallmarks of the human autopsy findings in Alzheimer's disease).

Transgenic mouse models overproducing mutant human APP reproduce important aspects of Alzheimer's disease, including amyloid plaques, neurodegeneration, and cognitive deficits. The first such model was generated with an alternatively spliced minigene encoding mutant hAPP⁶⁹⁵, hAPP⁷⁵¹, and hAPP⁷⁷⁰ directed by the platelet-derived growth factor (PDGF) B chain promoter. (Games, D. et al. (1995) Nature 373:523-527). This promoter directs transgene expression preferentially to neurons of the cortex, hippocampus, and cerebellum. Subsequent lines of mice using the same transgene but with additional mutations and higher levels of transgene expression developed diffuse and dense Aβ deposits, earlier signs of neurodegeneration, electrophysiological impairments, and behavioral deficits. Another transgenic mice strain, APP-T41 mice, also overproduces APP (specifically hAPP751^{V717I/K670M/N671L}) in neurons and these mice develop amyloid pathology, neurodegeneration, and cognitive deficits. Overexpression of wildtype human tau protein induces aggregation of abnormally phosphorylated tau and Alzheimer's disease-like neurofibrillary tangles. Transgenic mice with neuronal overexpression of mutant human tau protein (Prp-tauP301L mice) develop neurofibrillary tangles similar to the ones observed in Alzheimer's disease and suffer from locomotor deficits when they get old. These mice have also filamentous tau assemblies in oligodendrocytes and astrocytes, similar to glial inclusions in humans. Several additional transgenic models with different tau mutations associated have been generated and shown to develop tau filaments and/or neurofibrillary tangles.

Recently, a mouse model for Alzheimer's disease has been described that harbors three Alzheimer's disease mutant genes, tau^{P301L}, APP^{K670N,M671L}, and PS1^{M146V} and produces amyloid plaques, tangles, and synaptic transmission deficits. Transgenic mouse models that closely mimic the human pathology of Alzheimer's disease enable scientists and clinicians to understand the disease better. Any one or more of these transgenic mouse models can be used to evaluate the compositions and/or methods of treatment for Alzheimer's disease as described herein.

### Diagnosis of Alzheimer's Disease

As described herein, a group of AD-associated biomarkers, IL-1α, PDGF-BB, TNF-α, M-C SF, G-CSF, GNDF, eotaxin 2, MCP-3 PARC, AgRP, MSP-α, and BTC have been identified (listed in Table 1). These biomarkers demonstrate either increased or decreased protein plasma levels in individuals diagnosed with Alzheimer's disease as compared with controls (e.g., control individuals) as determined by antibody-based protein microarray analyses. This group of biomarkers may be used to assess cognitive function, to diagnose and aid in the diagnosis of Alzheimer's disease and/or to measure progression of Alzheimer's disease in an individual. The AD-associated biomarkers, IL-1α, PDGF-BB, TNF-α, M-CSF, G-CSF, GNDF, eotaxin 2, MCP-3 PARC, AgRP, MSP-α, and BTC, may be used individually or in combination for diagnosing or aiding in the diagnosis of Alzheimer's disease.

Provided are methods for the diagnosis of Alzheimer's disease or aiding the diagnosis of Alzheimer's disease in an individual by measuring the amount of one or more of the AD-associated biomarkers in a biological fluid sample, such as a peripheral biological fluid sample from the individual and comparing the measured amount with a reference (control) value for each AD-associated biomarker measured. The information obtained may be used to aid in the diagnosis or to diagnose Alzheimer's disease in an individual. Such methods may be used, for example, as an initial screening for Alzheimer's disease.

In some examples, methods for aiding diagnosis of Alzheimer's disease or diagnosing Alzheimer's disease and/or distinguishing Alzheimer's disease from other non-AD neurological diseases may comprise obtaining measured levels of one or more of the AD-associated biomarkers listed in Table 1 in a biological fluid sample from an individual, for example, a peripheral biological fluid sample from an individual and comparing those measure levels to reference levels. Accordingly, the present invention provides methods of aiding diagnosis or diagnosing Alzheimer's disease. Accordingly, the present invention provides methods of diagnosing Alzheimer's disease in an individual, said method comprising, a) detecting, measuring, and/or identifying one or more of the biomarkers selected from the group consisting of IL-1α, PDGF-BB, TNF-α, M-CSF, G-CSF, GNDF, eotaxin 2, MCP-3 PARC, AgRP, MSP-α, and BTC in a biological fluid sample from the individual; and/or b) comparing measured levels of any one or more of the biomarkers selected from the group consisting of IL-1α, PDGF-BB, TNF-α, M-CSF, G-CSF, GNDF, eotaxin 2, MCP-3 PARC, AgRP, MSP-α, and BTC in a biological fluid sample from the individual to an appropriate control.

The present invention also provides methods of diagnosing Alzheimer's disease in an individual, said method comprising, a) detecting, measuring, and/or identifying one or more of the biomarkers selected from the group consisting of IL-1α, TNF-α, M-CSF, eotaxin-2, MCP-3, PARC, and MSP-α in a biological fluid sample from the individual; and/or b) comparing measured levels of any one or more of the biomarkers selected from the group consisting of IL-1α, TNF-α, M-CSF, eotaxin-2, MCP-3, PARC, and MSP-α in a biological fluid sample from the individual to an appropriate control.

### Kits

The invention also provides kits for use in the instant methods. Kits of the invention may include one or more containers each comprising one or more polypeptides, agonists or antagonists. A kit may comprise polypeptides of IL-1α, PDGF-BB, TNF-α, M-CSF, G-CSF, GNDF, eotaxin 2, or MCP-3. In some examples, a kit comprises at least one polypeptide. In some examples, a kit comprises at least two polypeptides. In some examples, a kit comprises three or more polypeptides.

A kit may comprise agonists of IL-1α, PDGF-BB, TNF-α, M-CSF, G-CSF, GNDF, eotaxin 2, or MCP-3 and/or agonists of a receptor of IL-1α, PDGF-BB, TNF-α, M-CSF, G-CSF, GNDF, eotaxin 2, or MCP-3. In some examples, a kit comprises more than one agonist. A kits may comprise antagonists of PARC, AgRP, MSP-α or BTC or antagonists of a receptor of PARC, AgRP, MSP-α or BTC. In some examples, a kit comprises more than one antagonist. In some examples, a kit comprises a combination of polypeptides, agonists and antagonists.

In some examples, a kit further comprises instructions for use in accordance with any of the methods of the invention described herein, such as methods for administering to an individual i) at least one polypeptide of IL-1α, PDGF-BB, TNF-α, M-CSF, G-CSF, GNDF, eotaxin 2, or MCP-3; ii) at least one agonist of IL-1α, PDGF-BB, TNF-α, M-CSF, G-CSF, GNDF, eotaxin 2, or MCP-3; iii) at least one agonist of a receptor of IL-1α, PDGF-BB, TNF-α, M-CSF, G-CSF, GNDF, eotaxin 2, or MCP-3; iv) at least one antagonist of PARC, AgRP, MSP-α or BTC; or v) at least one antagonist of a receptor of PARC, AgRP, MSP-α or BTC. Instructions may be provided in printed form, on magnetic media, such as a CD or DVD, or in the form of a website address at which the instructions may be obtained.

The kits are in suitable packaging. Suitable packaging includes, but is not limited to, vials, bottles, jars, flexible packaging (e.g., sealed Mylar or plastic bags), and the like. In some examples, the kit comprises a container and a label or package insert(s) on or associated with the container. The label or package insert may indicate that the polypeptides, the agonists or the antagonists are useful for any of the methods described herein. Instructions may be provided for practicing any of the methods described herein.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed.

### Example 1

### Identification of Predictive Biomarkers

Using antibody-based protein microarrays, plasma protein levels for 120 cytokines, chemokines, growth factors, soluble receptors and hormone-like proteins were determined in biological fluid samples obtained from individuals diagnosed with Alzheimer's disease and control individuals as previously described in U.S. patent application publication No. 2005/0221348. As described in U.S. patent application publication No. 2005/0221348, biological fluid samples include peripheral biological fluid samples, blood, plasma and serum. The differences in protein levels were analyzed using a statistical algorithm that identifies a minimal set of markers that can discriminate and predict a certain “class” (R. Tibshirani et al. (2002) PNAS 99:6567-6572). With this method the data points are scaled to have a mean of 0 and a standard deviation of 1. The expression values are relative and are not absolute concentrations. The Tibhsirani, supra, method was used to undertake a predictive analysis of microarray data (PAM) and a group of proteins was identified that can be used as predictive AD-associated biomarkers. As an example of PAM, of 98 biological samples, for 65 samples (training set) 1 to 120 biomarkers were used to determine the numbers of predictor biomarkers and their error rate in predicting class for the samples; a 10 fold cross validation of the 65 samples in the training set was performed (this test is used to determine if the predictors identified in the training set are over-fitting; 1 to 120 biomarkers are used to determine predictors from randomly chosen 60 samples for lowest error rate in predicting class for 6 random samples, 10 times); the remaining 33 samples (test set) were used as an unbiased independent test to determine accuracy of biomarkers selected in training and cross-validation in predicting classes of samples in test set; the smallest number of biomarkers which had the lowest error rate for predicting class in training and 10-fold cross validation were identified as predictive biomarkers. This group included the following twelve biomarkers: interleukin 1α (IL-1α), platelet-derived growth factor beta chain (PDGF-BB), tumor necrosis factor-α (TNF-α), macrophage colony stimulating factor (M-CSF), granulocyte colony-stimulating factor (G-CSF), glial cell line-derived neurotrophic factor (GDNF), eotaxin 2, monocyte chemotactic protein 3 (MCP-3), pulmonary and activation-regulated chemokine (PARC), agouti-related protein (AgRP), macrophage stimulating protein-α (MSP-α) and betacellulin (BTC) and are listed in Table 1. The predictive accuracy of the 12 biomarkers was as follows: in cross-validation, 97% Sensitivity (31/32) samples were correct; 88% specificity (29/33) samples were correct, with a 92% accuracy. In the test set, 100% sensitivity (16/16) AD cases were correct; 94% specificity (16/17) controls were correct with a 97% accuracy. The 12 identified biomarkers appeared to be predictive across all stages of AD. Stages of AD as determined by for example, MMSE scores, are known in the art. Training set data, cross-validation data and test set data from PAM analysis of 1-120 biomarkers are shown in FIG. 1.

### Example 2

### Analysis of Alzheimer's Disease Samples

The biomarkers identified in the PAM analysis described in Example 1 and described herein were used in an unsupervised clustering analysis with the original 98 samples. The sample distribution among the AD and NDC samples is detailed in Table 5.

TABLE 5

| | Sample | Autopsy | Mean | MMSE |
|---|---|---|---|---|
| AD Stage | Number | Confirmed | MMSE | Range |
| Questionable or | 8 | 6 | 26.5 | 26-28 |
| Probable | | | | |
| Mild | 19 | 4 | 22 | 20-24 |
| Moderate | 17 | 7 | 14.9 | 11-19 |
| Severe | 4 | | 3.8 | 1-5 |
| Total | 48 | | 18.8 | 1-28 |
| Non-Demented | 50 | | 30 | 28-30 |
| Controls | | | | |

Using the biomarkers IL-1α, PDGF-BB, TNF-α, M-CSF, G-CSF, GNDF, eotaxin 2, MCP-3, PARC, AgRP, MSP-α, and BTC, the 98 samples were grouped resulting in 48 of 50 (96%) non-demented control samples clustered correctly and 45 of 48 (93.75%) Alzheimer's disease samples clustered correctly.

While the foregoing invention has been described by way of illustration and example for purposes of clarity of understanding, it will be apparent to one of skill in the art that various changes and modifications may be practiced without departing from the scope of the invention. For example, the invention has been described with human patients as the usual recipient, but veterinary use is also contemplated. Therefore, the foregoing description should not be construed as limiting the scope of the invention.

All publications, patents and published patent applications mentioned in this specification are hereby incorporated by reference into the specification in their entirety for all purposes.

1. A method for treating or preventing Alzheimer's disease (AD) in an individual, said method comprising modulating any one or more AD-associated biomarker(s) selected from the group consisting of interleukin-1α (IL-1α), platelet-derived growth factor-BB (PDGF-BB), tumor necrosis factor-α (TNF-α), macrophage colony-stimulating factor (M-CSF), granulocyte colony-stimulating factor (G-CSF), glial cell line-derived neurotrophic factor (GNDF), eotaxin 2, monocyte chemotactic protein-3 (MCP-3), pulmonary and activation-regulated chemokine (PARC), Agouti-related protein (AgRP), macrophage stimulating protein-α (MSP-α), and betacellulin (BTC) in a biological sample from said individual.

2. The method according to claim 1, wherein the modulation comprises increasing a level of at least one AD-associated biomarker selected from the group consisting of: IL-1α, PDGF-BB, TNF-α, M-CSF, G-CSF, GNDF, eotaxin-2 and MCP-3 in a biological sample from said individual.

3-4. (canceled)

5. The method according claim 1, wherein the modulation comprises decreasing a level of at least one AD-associated biomarker selected from the group consisting of: PARC, AgPR, MSP-α and BTC, in a biological sample from said individual.

6-7. (canceled)

8. The method according to claim 1, wherein the method comprises:
administering an agent which modulates monocyte/macrophage function in an amount sufficient to modulate at least one AD-associated biomarkers selected from the group consisting of: IL-1α, TNF-α, M-CSF, eotaxin-2, MCP-3, PARC and MSP-α.

9. A method for treating or preventing Alzheimer's disease in an individual, wherein said method comprises administering to said individual a therapeutically effective amount of a composition comprising at least one substance selected from the group consisting of:
a polypeptide of IL-1α, PDGF-BB, TNF-α, M-CSF, G-CSF, GNDF, eotaxin-2 or MCP-3;
a fragment or variant of IL-1α, PDGF-BB, TNF-α, M-CSF, G-CSF, GNDF, eotaxin-2 or MCP-3 that retains a biological activity;
an agonist of IL-1α, PDGF-BB, TNF-α, M-CSF, G-CSF, GNDF, eotaxin-2 or MCP-3;
an agonist of a receptor of IL-1α, PDGF-BB, TNF-α, M-CSF, G-CSF, GNDF, eotaxin-2 or MCP-3;
an antagonist of PARC, AgRP, MSP-α or BTC; and
an antagonist of a receptor of PARC, AgRP, MSP-α or BTC.

10. (canceled)

11. The method according to claim 9, wherein said agonist is selected from the group consisting of: a small molecule, an antibody, a biomarker mimic, a biomarker structural analog and a nucleic acid molecule.

12. The method according to claim 9, wherein said antagonist is selected from the group consisting of: a small molecule, an antibody, a biomarker structural analog and a nucleic acid molecule.

13. The method according to any claim 9, wherein said composition comprises at least one polypeptide, or a fragment thereof, in an amount sufficient to result in a significant increase in a level of said polypeptide in a biological fluid sample from said individual.

14. The method according to any claim 9, wherein said composition comprises an agonist, in an amount sufficient to result in a significant increase in a level of IL-1α, PDGF-BB, TNF-α, M-CSF, G-CSF, GNDF, eotaxin-2 or MCP-3 in a biological sample from said individual.

15. The method according to any claim 9, wherein said composition comprises an antagonist, in an amount sufficient to result in a significant decrease in a level of PARC, AgRP, MSP-α and/or BTC in a biological sample from said individual.

16. A method according to any claim 9, wherein the treating comprises alleviating at least one symptom of Alzheimer's disease.

17-18. (canceled)

19. The method according to any claim 9, wherein said individual has at least one risk factor for Alzheimer's disease, wherein said risk factor is selected from the group consisting of: diagnosis of mild cognitive impairment, advanced age, family history, genetics, Down syndrome, history of head injury, exposure to environmental toxins and low education level.

20. The method according to any claim 9, wherein said preventing comprises a method selected from the group consisting of: halting the onset of AD, reducing a risk of development of AD, reducing an incidence of AD, delaying an onset of AD, reducing development of symptoms of AD, and delaying an onset of symptoms of AD.

21. A method for delaying the development of Alzheimer's disease in an individual with Alzheimer's disease or at risk of developing Alzheimer's disease, the method comprising:
a) detecting in a biological sample an elevated level of a biomarker selected from the group consisting of PARC, AgRP, MSP-α and BTC, or a reduced level of a biomarker selected from the group consisting of IL-1α, PDGF-BB, TNF-α, M-CSF, G-CSF, GNDF, eotaxin-2 and MCP-3; and
b) administering a therapeutically effective amount of a composition comprising at least one substance selected from the group consisting of:
a polypeptide of IL-1α, PDGF-BB, TNF-α, M-CSF, G-CSF, GNDF, eotaxin-2 or MCP-3;
a fragment or variant of IL-1α, PDGF-BB, TNF-α, M-CSF, G-CSF, GNDF, eotaxin-2 or MCP-3 that retains a biological activity;
an agonist of IL-1α, PDGF-BB, TNF-α, M-CSF, G-CSF, GNDF, eotaxin-2 or MCP-3;
an agonist of a receptor of IL-1α, PDGF-BB, TNF-α, M-CSF, G-CSF, GNDF, eotaxin-2 or MCP-3;
an antagonist of PARC, AgRP, MSP-α or BTC; and
an antagonist of a receptor of PARC, AgRP, MSP-α or BTC.

22. The method according to claim 21, wherein the detection comprises use of an antibody-based array wherein the array comprises antibodies specific for one or more polypeptides selected from the group consisting of: IL-1α, PDGF-BB, TNF-α, M-CSF, G-CSF, GNDF, eotaxin-2, MCP-3, PARC, AgRP, MSP-α and BTC.

23. The method according to any claim 21, wherein the biological sample is a peripheral biological fluid sample selected from the group consisting of blood, plasma and serum.

24. A method according to claim 1, wherein the treating comprises alleviating at least one symptom of Alzheimer's disease.

25. The method according to claim 1, wherein said individual has at least one risk factor for Alzheimer's disease, wherein said risk factor is selected from the group consisting of: diagnosis of mild cognitive impairment, advanced age, family history, genetics, Down syndrome, history of head injury, exposure to environmental toxins and low education level.

26. The method according to claim 1, wherein said preventing comprises a method selected from the group consisting of: halting the onset of AD, reducing a risk of development of AD, reducing an incidence of AD, delaying an onset of AD, reducing development of symptoms of AD, and delaying an onset of symptoms of AD.


SEQUENCE LISTING (12 sequences)

SEQ ID NO: 1 - protein, 271 aa, Homo sapien
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 2 - protein, 241 aa, Homo sapien
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 3 - protein, 233 aa, Homo sapien
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 554 aa, Homo sapien
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 5 - protein, 207 aa, Homo sapien
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 6 - protein, 211 aa, Homo sapien
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 7 - protein, 119 aa, Homo sapien
MAGLMTIVTSLLFLGVCAHHIIPTGSVVIPSPCCMFFVSKRIPENRVVSYQLSSRSTCLKAGVIFTTKKGQQFCGDPKQEWVQRYMKNLDAKQKKASPRARAVAVKGPVQRYPGNQTTC

SEQ ID NO: 8 - protein, 99 aa, Homo sapien
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 9 - protein, 89 aa, Homo sapien
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 10 - protein, 132 aa, Homo sapien
MLTAAVLSCALLLALPATRGAQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT

SEQ ID NO: 11 - protein, 711 aa, Homo sapien [sequence omitted]

SEQ ID NO: 12 - protein, 178 aa, Homo sapien
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA